(19) United States
(12) Patent Application Publication
(10) Pub. No.: US 2019/0142893 A1
(43) Pub. Date: May 16, 2019

(54) Electron, Radical, or Reactive Oxygen Species-Scavenging Agent Based Therapy of Inborn Errors of Fatty Acid Oxidation and Oxidative Phosphorylation

(71) Applicant: University of Pittsburgh- Of the Commonwealth System of Higher Education, Pittsburgh, PA (US)

(72) Inventors: Al-Walid A. Mohsen, Gibsonia, PA (US); Gerard Vockley, Pittsburgh, PA (US); Yudong Wang, Cheswick, PA (US); Peter Wipf, Pittsburgh, PA (US)

(21) Appl. No.: 16/098,322

(22) PCT Filed: May 5, 2017
(86) PCT No.: PCT/US2017/031312

§ 371 Date: Nov. 1, 2018

Related U.S. Application Data
(60) Provisional application No. 62/332,894, filed on May 6, 2016.

(51) Int. Cl. A61K 38/06; A61P 3/00; A61P 9/00; A61P 19/00; A61K 31/4468

(52) U.S. Cl. CPC A61K 38/06; A61P 3/00; A61K 31/4468; A61P 19/00; A61P 9/00

(57) ABSTRACT

A method is provided for treating a fatty acid oxidation metabolic condition, such as an inborn error of fatty acid oxidation or oxidative phosphorylation, and/or hypoglycemia, rhabdomyolysis, or cardiomyopathy in a patient. A mitochondrial-targeted electron, radical, or ROS-scavenging agent is administered to the patient in an amount effective to treat, mitigate or prevent any fatty acid oxidation metabolic condition, such as inborn errors of fatty acid oxidation or oxidative phosphorylation, and/or hypoglycemia, rhabdomyolysis, or cardiomyopathy in a patient.

### CROSS REFERENCE TO RELATED APPLICATIONS

This application claims the benefit of U.S. Patent Application No. 62/332,894, filed May 6, 2016, which is incorporated herein by reference in its entirety.

### STATEMENT REGARDING FEDERAL FUNDING

This invention was made with government support under Grant Nos. GM067082, A1068021 and DK078775, awarded by the National Institutes of Health. The government has certain rights in the invention.

Mitochondrial fatty acid oxidation is a complex process involving transport of activated acyl-CoA moieties into the mitochondria, and sequential removal of 2 carbon acetyl-CoA units. It is the main source of energy for many tissues including heart and skeletal muscle and is critically important during times of fasting or physiologic stress. When the body's glycogen stores are depleted, long-chain fatty acids are mobilized from adipose tissue and taken up by liver and muscle cells. While short- and medium-chain fatty acids (C₄ to C₁₂) diffuse freely across plasma and mitochondrial membranes, the transport of longer chain species (C₁₄ to C₂₀) depends at least in part on active transport, a high-affinity mechanism of major physiological importance in skeletal muscle, liver, and adipocytes. Two additional enzymatic steps are necessary for the complete oxidation of mono- and di-unsaturated fatty acids, 2,4 dienoyl-CoA reductase and an enoyl-CoA isomerase, which allow for the complete oxidation of physiologically abundant fatty acids such as linoleate (C₁₈:2) and oleate (C18:1). Each cycle of the pathway produces a molecule of acetyl-CoA and a fatty acid with two fewer carbons. Under physiological conditions, the latter re-enters the cycle until it is completely consumed. In peripheral tissues, the acetyl-CoA is terminally oxidized in the Krebs cycle for ATP production. In the liver, the acetyl-CoA from fatty acid oxidation can instead be utilized for the synthesis of ketones, 3-hydroxybutyrate, and acetoacetate, which are then exported for final oxidation by brain and other tissues. At least 25 enzymes and specific transport proteins are responsible for carrying out the steps of mitochondrial fatty acid metabolism, some of which have only recently been recognized. Of these, defects in at least 22 have been shown to cause disease in humans.

Most patients with fatty acid oxidation defects are now identified through newborn screening by tandem mass spectrometry of carnitine esters in blood spots. Unscreened patients can present throughout life. In the first week of life, cardiac arrhythmias, hypoglycemia, sudden death, and occasionally with facial dysmorphism and malformations, including renal cystic dysplasia are seen. Symptoms later in infancy and early childhood may relate to the liver or cardiac or skeletal muscle dysfunction, and include fasting or stress-related hypoketotic hypoglycemia or Reye-like syndrome, conduction abnormalities, arrhythmias or dilated or hypertrophic cardiomyopathy, and muscle weakness or fasting- and exercise-induced rhabdomyolysis. Adolescent or adult onset muscular symptoms, including rhabdomyolysis, and cardiomyopathy predominate. Diagnosis can usually be established even when the patient is asymptomatic, though analysis of samples during acute illness can uncover some mild cases.

The most important single diagnostic test is analysis of acylcarnitine esters in serum, plasma, or dried blood spots by tandem mass spectroscopy, which will identify characteristic compounds in many of these conditions. Other tests that may be useful include urine organic acids and acylglycines, free and total carnitine in serum and urine, and enzyme assays or flux studies in leukocytes or fibroblasts. Treatment of the acute encephalopathy of hypoketotic hypoglycemia is by intravenous glucose and l-carnitine. Long-term therapy involves replenishing carnitine stores with l-carnitine, and preventing hypoglycemia. In some cases this can be done by providing a snack, glucose polymers, or uncooked cornstarch before bedtime, but in others requires continuous intragastic feeding. Supplementation with medium chain triglyceride (MCT) oil provides a fat source that can be utilized by patients with long chain defects. Safe and effective treatments for fatty acid oxidation metabolic conditions are needed, including remedies for correcting the molecular cause and ones to alleviate the pathophysiological consequences.

### SUMMARY

According to one aspect of the invention, a method of treating a fatty acid oxidation or respiratory chain metabolic condition, and/or hypoglycemia, rhabdomyolysis, lactic acidosis, hyperammonemia, skeletal myopathy, hypotonia, fatiguabilty, or cardiomyopathy, e.g., arising from a fatty acid oxidation or respiratory chain metabolic condition, in a patient, is provided. The method comprises administering to the patient an amount of a mitochondria-targeting electron, radical, or ROS-scavenging agent effective to treat the fatty acid oxidation or respiratory chain metabolic condition, and/or hypoglycemia, rhabdomyolysis, lactic acidosis, hyperammonemia, skeletal myopathy, hypotonia, fatiguabilty, or cardiomyopathy, e.g., arising from a fatty acid oxidation or respiratory chain metabolic condition, in a patient. The fatty acid oxidation or respiratory chain metabolic condition, according to various aspects, may be an inborn error of fatty acid oxidation or oxidative phosphorylation, wherein for example, the inborn error of fatty acid oxidation or oxidative phosphorylation is chosen from defects in the following enzymes: carnitine palmitoyltransferase (CPT) I; CPT II; carnitine-acylcarnitine translocase (CACT); very long-chain acyl-CoA dehydrogenase (VLCAD); medium-chain acyl-CoA dehydrogenase (MCAD); and long-chain hydroxyacyl-CoA dehydrogenase (LCHAD).

In other aspects, a mitochondria-targeting electron, radical, or ROS-scavenging agent according to any aspect provided herein, is provided for use in preparing a medicament for use in treatment of a fatty acid oxidation or respiratory chain metabolic condition, and/or hypoglycemia, rhabdomyolysis, lactic acidosis, hyperammonemia, skeletal myopathy, hypotonia, fatiguabilty, or cardiomyopathy in a patient. In further aspects, a mitochondria-targeting electron, radical, or ROS-scavenging agent according to any aspect provided herein, is provided for treatment of a fatty acid oxidation or respiratory chain metabolic condition, and/or hypoglycemia, rhabdomyolysis, lactic acidosis, hyperammonemia, skeletal myopathy, hypotonia, fatiguabilty, or cardiomyopathy in a patient.

### BRIEF DESCRIPTION OF THE DRAWINGS

FIG. 1 provides structures for examples of mitochondria-targeting electron, radical and reactive oxygen species (ROS) scavengers, as described below.

FIG. 2 provides an exemplary synthesis scheme for JP4-039.

FIGS. 3A and 3B show the effect of exposure to different concentrations of H2O2 on the efficiency of PCR amplification of mtDNA and nDNA. The nDNA and mtDNA damage was assessed after a 1 h exposure in serum-free medium containing 0, 100, 200, 300, and 400 μM H₂O₂ at 37° C. DNA isolated from these cells was used as template for PCR to amplify a 12 kb mtDNA section (FIG. 5A, top) and a 13.4 kb section of nDNA (FIG. 3A, bottom). (FIG. 3B) Quantification of each fragment by densitometric scanning.

FIGS. 4A and 4B. ROS generation in cultured cells after exposure to 200 μM H₂O₂ one hour/day for four days was measured by ESR. FIG. 4A(A) Mitochondria were suspended in PBS containing 5 mM glutamate/malate, 100 μM NADH, and 100 mM succinate, and 200 mM DMPO. Spectrum FIG. 4A(A.a) base line; FIG. 4A(A.b) untreated, and FIG. 4A(A.g) untreated and grown under normal condition for 20 days. The mitochondria were separated from the cells that were exposed four times to 200 μM H₂O₂ and then grown in a normal condition for FIG. 4A(A.c) 3 days, FIG. 4A(A.d) 8 days, FIG. 4A(A.e) 12 days, and FIG. 4A(A.f) 20 days. FIG. 4A(B). The ROS production in the oxidative exposure and following 20 days grown under normal culture condition as described in FIG. 4A(A.f) was tested. FIG. 4A(B.a) in the presence of 25 μM DBH2, a substrate for complex III and FIG. 4A(B.b) in the presence of 100 μM NADH, a substrate for complex I. (FIG. 4B) The ESR signal intensity is highlighted to show the change in the offspring of oxidative exposure cells.

FIG. 5. Nuclear DNA fragmentation. Total DNA was extracted from cells (A) untreated on day 1, (B) 5 days after oxidative exposure, (C) 15 days after oxidative exposure and (D) untreated cell harvested at day 15 and 0.7 μg were loaded on a 1% agarose gel. Arrows indicate the position of the DNA fragments characteristic of early apoptosis.

FIG. 6. Flow cytometry analysis of H₂O₂ treated cells. (A) Cells were doubly labeled with 7 AAD and PhiPhilux-G₁D₂. The mean fluorescent intensity of PhiPhilux-G₁D₂ in lower right quadrant (7 AAD negative) and represents apoptotic cells. (B) A histogram of PhiPhilux-G₁D₂ of untreated cells. The mean fluorescence intensity was ˜110. (C) A histogram of PhiPhilux-G₁D₂ 20 days after exposure to H₂O₂. The mean fluorescence intensity increased to 308.

FIGS. 7A and 7B. MtDNA damage by internally generated ROS in offspring of oxidative exposure cells. PCR of a 12 kb mtDNA fragment (Table I and Scheme 1) was used to estimate the damage of mtDNA in cells treated with 200 μM H₂O₂ at various times after the exposure. (FIG. 7A, lane 1) Untreated cells, day 1. (FIG. 7A, line 2) Untreated, day 15. (FIG. 7A, lane 3) 3 days after exposure, and (FIG. 7A, lane 4) 20 days after exposure. Sizes on the right side of the gel indicate the main products of mtDNA fragments produced at 3 and 20 days. FIG. 7B. Quantification of the 12 kb fragment in the gel lanes shown in FIG. 7A.

FIGS. 8A and 8B. The susceptibility of mtDNA to internal generated ROS is region specific. Oxidative damage of different sections of mtDNA caused by internal generated ROS was tested by PCR. Five pairs of primers were designed to amplify the Dloop (line 1), 12S/16S (line 2), ND1-2 (line 3), COX (line 4), and ND3-6 (line 5). FIG. 8A. The five DNA fragments amplified were isolated from untreated cells, 5 days after oxidative stress, and 20 days after oxidative stress. Quantification of each fragment by scanning densitometry is shown in FIG. 8B. The amount of DNA product generated from untreated cells was defined as 100%.

FIG. 9. Enzyme Activity of the ETC is reduced in cells exposed to oxidative stress. Enzyme activity of complexes I, III, and IV of ETC was tested at various times after exposure to 200 μM H₂O₂. Mitochondria were separated from cells 3, 8, 12 and 20 days after exposure. The enzyme activities of the untreated cells were defined as 100%, and activity at each day post H₂O₂ exposure is presented as a percentage as compared with untreated cells. Symbols are: (•) complex I, (♦) complex III, and (▴) complex IV.

FIG. 10. Superoxide production is increased in VLCAD cells (Fb671) as compared to wild type and it is decreased to wild type levels after incubation of cells with JP4-039.

FIG. 11. Basal respiration profile (A) and respiratory reserve capacity (B) of WT (wild type, control cell line) and Fb671 (VLCAD-deficient) fibroblasts cultured in normal media. OCR (oxygen consumption rate). ****P<0.0001, compared to WT (Mann-Whitney U test).

FIG. 12. Basal respiration profile (A) and respiratory reserve capacity (B) of WT (wild type, control cell line) and Fb671 (VLCAD-deficient) fibroblasts cultured in medium without glucose for 72 h. OCR (oxygen consumption rate). ****P<0.0001, compared to WT (Mann-Whitney U test).

FIG. 13. Basal respiration profile (A) and respiratory reserve capacity (B) of WT (wild type, control cell line) and Fb342 (MCAD-deficient) fibroblasts cultured in normal media. OCR (oxygen consumption rate). ****P<0.0001, compared to WT (Mann-Whitney U test).

FIG. 14. Basal respiration profile (A) and respiratory reserve capacity (8) of WT (wild type, control cell line), Fb342 and Fb752 (MCAD-deficient) fibroblasts cultured in medium without glucose for 72 h. OCR (oxygen consumption rate). ****P<0.0001, compared to WT (Mann-Whitney U test).

FIG. 15. ATP production of WT (wild type, control cell line) and Fb671 (VLCAD-deficient) fibroblasts cultured in normal media (A) or medium without glucose for 48 h (B). *P<0.05, compared to WT (Student t test).

FIG. 16. ATP production of WT (wild type, control cell line) and Fb342 (MCAD deficient) fibroblasts cultured in normal medium (A) or medium without glucose for 48 h (B). *P<0.05, compared to WT (Student t test).

FIG. 17. Immunoblot for VLCAD, ND6 (complex I subunit), VDAC1 (porin) and MFN 1 (mitofusin 1). Protein levels were measured in mitochondrial fractions from control (WT) and VLCAD deficient fibroblasts cultured in normal medium or medium without glucose for 48 h.

FIG. 18. Mitochondrial mass of WT (wild type, control cell line) and Fb671 (VLCAD-deficient) fibroblasts cultured in normal medium (A) or medium without glucose for 4 Sh (B).

FIG. 19. Mitochondrial mass of WT (wild type, control cell line) and Fb342 (MCAD-deficient) fibroblasts cultured in normal medium (A) or medium without glucose for 48 h (8).

FIG. 20. Superoxide production of WT (wild type, control cell line) and Fb671 (VLCAD-deficient) fibroblasts cultured in normal medium (A) or medium without glucose for 48 h (B). Data reported in AFU and normalized by number of events (cells).

FIG. 21. Superoxide production of WT (wild type, control cell line) and Fb671 (VLCAD-deficient) fibroblasts cultured in normal medium (A) or medium without glucose for 48 h (B). Data reported in AFU and normalized by number of events (cells).

FIG. 22. Superoxide production of WT (wild type, control cell line) and Fb671 (VLCAD-deficient) fibroblasts cultured in medium without glucose for 48 h and treated with N-acetylcysteine (A) for 24 h, Bezafibrate (B) for 48 h and JP4-039 (C) for 24 h. Data reported in AFU and normalized by number of events (cells).

FIG. 23. (A) Schematic diagram delineating functional effects of electron transport chain inhibitors on mitochondrial respiration. The experiment involves measuring the oxygen consumption rate (OCR) at the resting state (basal respiration) followed by injection of oligomycin (inhibitor of ATP synthase), and the drop in the OCR represents ATP turnover. Subsequent injection of FCCP dissipates the proton gradient and allows maximum respiration. The rise in OCR upon FCCP addition represents mitochondrial spare reserve capacity. Finally, a cocktail of rotenone and antimycin A are added to completely disable the electron transport chain, inhibiting the total mitochondrial respiration. The remaining OCR after complete inhibition of the electron transport chain represents non-mitochondrial respiration. (B) The OCR difference between oligomycin- and rotenone and antimycin A-responsive OCR reflects proton leak. Oxygen consumption rate (OCR) of Fb671, Fb773, Fb774, Fb777 and Fb780 fibroblasts cultured in without glucose for 72 h. Data are means±SD. ****P<0.0001, compared to WT (t test for unpaired samples).

FIG. 24. Effect of JP4-039 on basal respiration (A-C) and reserve capacity (D-F) of Fb671, Fb773 and Fb834 fibroblasts cultured in media without glucose for 72 h. VLCAD-deficient cells were exposed to JP4-039 (40 nM or 200 nM) during 24 h. Data are means±SD. ****P<0.0001, compared to WT; ^{#}P<0.05, ^{##}P<0.01, P<0.001 ^{###}P<0.0001, compared to Fb671, Fb773 or Fb834 (Tukey multiple range test).

FIG. 25. Seahorse measurements of oxygen consumption in VLCAD deficient cells (FB671) cultured in media without glucose. Treatment with XJB-5-131 at 200 nM improved basal respiration, and treatment at 40 nM and 200 nM improves the reserved capacity of deficient but not wild type cells.

FIG. 26. Reactive species production in Fb671, Fb773, Fb834 and Fb833 fibroblasts cultured in media with (A-C) or without glucose for 48 h (D-F). Data are means f SD. **P<0.01, ***P<0.001, compared to WT (t test for unpaired samples).

FIG. 27. Effect of N-acetylcysteine (NAC), Bezafibrate (Bez), Resveratrol (Resv), MitoQ and Trolox on superoxide levels in Fb671 or Fb773 fibroblasts cultured in media without glucose for 48 h. VLCAD-deficient cells were exposed to the different compounds during 24 or 48 h. Data are means±SD. **P<0.05, **P<0.01, ***P<0.001, ****P<0.0001, compared to WT; ^{##}P<0.01, ^{####}P<0.0001, compared to VLCAD-deficient fibroblasts (Tukey multiple range test).

FIG. 28. Effect of JP4-039 on superoxide levels in Fb671, Fb773, Fb834 and Fb833 fibroblasts cultured in media without glucose for 48 h. VLCAD-deficient cells were exposed to JP4-039 (40 or 200 nM) during 24 h. Data are means f SD. *P<0.05, **P<0.01, ***P<0.001, compared to WT; ^{#}P<0.05, ^{##}P<0.01, compared to Fb671, Fb773, Fb834 or Fb833 (Tukey multiple range test).

FIGS. 29A-29C. Nrf2 protein content in nucleus (FIG. 29A) and cytosol (FIG. 29B) prepared from Fb671 fibroblasts, normalized by the content of the proteins lamin B1 (A) or β-actin (FIG. 29B). Fibroblasts were cultured in media without glucose for 48 h and treated with DMSO or JP4-039 for 24 h. The representative images (FIG. 29C) show colocalization of Nrf2 protein visualized with green (in original) fluorescently tagged antibody and nuclei visualized with DAPI staining as yellow (white arrows). Calibration bar indicates 50 μm.

FIGS. 30A-30C. NF-κB protein content in nucleus (FIG. 30A) and cytosol (FIG. 30B) prepared from Fb671 fibroblasts, normalized by the content of the proteins lamin B1 (FIG. 30A) or β-actin (FIG. 30B). Fibroblasts were cultured in media without glucose for 48 h and treated with DMSO or JP4-039 for 24 h. The representative images (FIG. 30C) show colocalization of Nrf2 protein visualized with green (in original) fluorescently tagged antibody and nuclei visualized with DAPI staining as yellow (white arrows). Calibration bar indicates 50 μm.

FIGS. 31A and 31B. Fatty acid oxidation (FAO) flux (FIG. 31A) and very long-chain acyl-CoA dehydrogenase (VLCAD) protein content (FIG. 31B) in Fb671 fibroblasts cultured in media with or without glucose for 48 h. FAO flux was measured in fibroblasts cultured in a 24-well plate. VLCAD content was measured in mitochondria prepared from fibroblasts. Data are means±SD. *P<0.05, **P<0.01, compared to WT; ^{#}P<0.05, compared to Fb671 (Tukey multiple range test).

FIGS. 32A and 32B. Cell viability (FIG. 32A) and VCACI/porin protein content (FIG. 32B) in Fb671 fibroblasts cultured in media with or without glucose for 48 h. Cell viability were measured in fibroblasts cultured in a 96-well plate. VDAC₁/porin content was measured in mitochondria prepared from fibroblasts. Data are means±SD. **P<0.01, ***P<0.001, compared to WT (t test for unpaired samples).

FIGS. 33A and 33B. Citrate synthase activity in Fb671 (FIG. 33A) and Fb773 (FIG. 33B) fibroblasts cultured in media with or without glucose for 48 h. Data are means f SD. *P<0.05, compared to WT (t test for unpaired samples).

FIG. 34. Seahorse measurements of oxygen consumption in isolated LCHAD deficient cells.

FIG. 35. Seahorse measurements of oxygen consumption in MTP deficient cells.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the terms “patient” or “subject” refer to members of the animal kingdom including but not limited to human beings.

“Conditions Associated with Errors in Mitochondrial Fatty Acid Oxidation and Oxidative Phosphorylation” include deficiencies in expression and/or activity of various mitochondrial enzymes, including enzymes related to mitochondrial uptake of fatty acids and enzymes relating to β-oxidation, and include mitochondrial respiratory chain deficiencies. The following describes examples of such conditions.

Mitochondrial uptake of fatty acids longer than C₁₀₋₁₂ requires esterification to an acyl-CoA, and the concerted action of carnitine palmitoyltransferases (CPT) I and II, and carnitine-acylcarnitine translocase (CACT). CPT I in the outer mitochondrial membrane first transfers the acyl moiety from CoA to carnitine, and the translocase moves the acylcarnitine ester across the inner membrane in exchange for free carnitine. CPT II in the inner membrane then reconstitutes the CoA esters, which enter the β-oxidation spiral.

Severe deficiency of liver CPT I is rare but more frequent milder variants have been identified in geographically restricted populations. Severe symptoms include episodic hypoketotic hypoglycemia beginning in infancy and multi-organ-system failure. Cardiac symptoms are not present. Creatine kinase levels in blood are elevated in acute episodes. Organic aciduria is not prominent in this disorder, but hyperammonemia may be present. Mild CPTI deficiency is found in high frequency in first nation populations in Canada and Alaska where it is most frequently identified through newborn screening.

Deficiency of the CACT was initially reported in newborns who had a nearly uniform poor outcome, presenting with severe hypoketotic hypoglycemia and cardiac arrhythmias and/or hypertrophy. All have had a grossly elevated acylcarnitine to free carnitine ratio, while dicarboxylic aciduria was reported in one. Patients with a more benign clinical course have since been identified who have responded well to modest carnitine supplementation and dietary therapy. Two affected sibs have been reported where the younger sib was prospectively treated and has not developed any sequelae 2 years later. It appears that these patients have a higher level of residual enzyme activity than the more severely affected patients. Specific diagnosis of this disorder can be made via direct enzyme or molecular analysis.

CPT II deficiency is the most common of this group of disorders. It classically presents in late childhood or early adulthood as episodes of recurrent exercise or stress induced myoglobinuria. Episodes can be severe enough to lead to acute renal failure. Patients are typically well between episodes. There is no tendency to develop hypoglycemia. Weakness and muscle pain are reported. The characteristic diagnostic finding in these patients is a low total plasma carnitine level with increased acylcarnitine fraction and no dicarboxylic aciduria. Long chain acylcarnitines may be elevated. A more severe variant of CPT II deficiency presenting with symptoms similar to severe CACT deficiency has been described. In these patients, the presenting symptoms were neonatal hypoglycemia, hepatomegaly, and cardiomyopathy. Several polymorphic variants in the CPT gene have been associated with an adverse neurologic outcome in influenza encephalitis in Japan.

The serum acylcarnitine profile is usually normal in CPT I deficiency, but acylcarnitine levels are low. CPT II and translocase deficiency can be identified but not distinguished from each other by biochemical testing, both showing elevated C₁₆ esters. The acylcarnitine profile may be normal in milder disease. Urine organic acids are either normal or show mild dicarboxylic aciduria. Blood amino acids are usually normal. Free carnitine in serum is 2 to 3 times normal in CPT I deficiency, and is very low in CPT II and translocase deficiency. All three enzymes can be assayed in fibroblasts and leukocytes.

Acute episodes of hypoketotic hypoglycemia should be treated with intravenous glucose-containing fluids to provide at least 8-10 mg/kg/min of glucose. Treatment of hyperammonemia may require dialysis. Ammonia conjugating agents are usually not needed as the hyperammonemia reverses with correction of the underlying metabolic process. Prevention of fasting is the mainstay of therapy in all three disorders and continuous intragastic feeding may be necessary in severe disease. Carnitine supplementation is not usually effective But should be considered when free carnitine is extremely low. Bezafibrate has been shown to induce fatty acid oxidation in cells and improve flux through fatty acid oxidation in cells from patients with residual CPT2 activity. A subsequent small trial (6 patients) indicated improvement over a 3 year period of treatment. Expansion of this therapy to other long chain fatty acid oxidation disorders may be possible.

All three enzyme defects are inherited as autosomal recessive traits, and the genes CPTIA, CPT2, and SLC25A20 (the gene for the translocase) have been localized to chromosomes 11 (11q13), 1 (1p32), and 3 (3p31.21), respectively. Disease-causing mutations have been identified in all three genes, with a relatively common mutation has present in the late onset muscular form of CPT II deficiency, and mild CPTI deficiency in the Hutterite population. Numerous coding polymorphisms of unknown significance have been identified in the CPTI gene. Prenatal diagnosis by mutation analysis or enzyme assay on amniocytes is possible in all three conditions.

Once in the mitochondrial matrix, acyl-CoA esters enter the β-oxidation spiral in which a series of four reactions successively removes two-carbon fragments of acetyl-CoA. All of the enzymes of β-oxidation have distinct (and often overlapping) substrate chain-length specificities. For instance, different FAD-containing dehydrogenases oxidize very long-chain (C₁₂₋₂₄), long-chain (C₆₋₂₀), medium-chain (C₁₋₁₄), and short-chain (C₄₋₆) acyl-CoAs, and similar specificities exist for the hydratases, hydroxyacyl-CoA dehydrogenases, and thiolases. Inherited defects in almost all of these enzymes have been described. As a rule, defects in long-chain specific enzymes block oxidation more completely and cause more severe clinical diseases than do deficits in the medium and short chain specific enzymes. Although most of these conditions were originally thought to be rare, defects in very long-chain acyl-CoA dehydrogenase (VLCAD), medium-chain acyl-CoA dehydrogenase (MCAD), and long-chain hydroxyacyl-CoA dehydrogenase (LCHAD) are among the most common metabolic defects identified though newborn screening with tandem mass spectrometry. Patients who were originally reported with long-chain acyl-CoA dehydrogenase (LCAD) deficiency have all in fact been subsequently shown to have defects of VLCAD. Thus no bone fide patients with LCAD deficiency are known to exist.

Very long-chain acyl-CoA dehydrogenase deficiency can present in the newborn period with arrhythmias and sudden death, or with hepatic, cardiac, or muscle presentations later in infancy or childhood. The hepatic presentation is characterized by fasting-induced hypoketotic hypoglycemia, encephalopathy, and mild hepatomegaly, often with mild acidosis, hyperammonemia, and elevated liver transaminases. Some present with arrhythmias or dilated or hypertrophic cardiomyopathy in infancy or childhood, and some with adolescence onset of exercise or fasting-induced muscle pain, rhabdomyolysis, elevated creatine phosphokinase, and myoglobinuria. The disorder is inherited as an autosomal recessive trait. Tendency to develop hypoglycemia decreases with age, but low grade, chronic rhabdomyolysis with acute exacerbations are common.

ACAD9 (VLCAD) deficiency has been reported with two distinct phenotypes. In the first publication, patients had severe recurrent hypoglycemia with hepatocellular failure reversible with administration of intravenous glucose. One set of sibs also had cardiomyopathy. All reported patients appeared to have null mutations as indicated by lack of enzyme antigen. The deficiency has also been reported in patients with deficiency of complex I of the respiratory chain apparently due to a second function for ACAD9 as a complex I assembly or stability factor. All of these patients have had point mutations, a finding that may be integral to determining the clinical picture. In fact, both phenotypes can be explained by the existence of a multi-functional protein complex within mitochondria that contains both the respiratory chain and fatty acid oxidation enzymes providing close physical and functional relationships between the two pathways.

Analysis of serum acylcarnitines by tandem mass spectrometry usually shows elevations of saturated and unsaturated C₁₄₋₁₈ esters in VLCAD deficiency, even between episodes. Organic acid analysis during acute episodes often shows C₆, C₈, and C₁₀ dicarboxylic aciduria, but because these acids can also be seen when physiological ketosis is resolving, or following the intake of medium-chain triglycerides, this will not raise suspicion of disease unless C₁₂ and C₁₄ dicarboxylic acids are also present. Free carnitine in serum is usually low. If necessary, enzyme deficiency can be demonstrated in fibroblasts or leukocytes. Molecular testing is readily available. VLCAD deficiency is now most frequently diagnosed by newborn screening with tandem MS. No consistent specific biochemcial markers in blood or urine have been identified in patients with ACAD9 deficiency. Liver acylcarnitine profile has been reported to be abnormal with an excess of unsaturated compared to saturated species.

Acute management of VLCAD deficiency involves administration of high infusion of high rates of glucose-containing intravenous fluids to give 8-10 mg/kg/min of glucose. Chronic management is somewhat controversial. Avoiding fasting and maintaining a high carbohydrate intake are clearly indicated, and continuous intragastic feeding may be necessary to achieve this goal especially overnight. Medium-chain triglycerides, whose oxidation does not involve VLCAD, can be administered to provide calories but should not be used until a diagnosis of MCAD deficiency has been excluded. However, safe fasting intervals, the use of oral carnitine, and substitution in the diet of the experimental medium chain oil triheptanoin are more controversial. As with CPT2 deficiency, bezafibrate has been suggested as a possible means of increasing activity in patients with partially stable mutations and residual enzyme activity. Treatment of ACAD9 deficiency remains uncertain due to its infrequency. Institution of high glucose infusion is warranted if hypoglycemia or elevated liver enzymes are elevated, but the need for chronic management when well has not been demonstrated.

The ACADVL gene has been cloned and localized to chromosome 17 (17p13), and although several disease-causing mutations are known there is no single prominent mutation. In general, the more severe defects cause the most severe and early presenting clinical disease. Prenatal diagnosis is possible through enzyme assay in cultured amniocytes, by demonstrating abnormal metabolism of stable isotopically labeled palmitate by amniocytes, and by mutation analysis. The ACAD9 gene is on chromosome 3q21.3. There have been no reported cases of prenatal diagnosis.

The most common of the fatty acid oxidation disorders, MCAD deficiency, historically most frequently presented during the first 2 years of life with episodes of fasting-induced vomiting, hepatomegaly, hypoketotic hypoglycemia, and lethargy progressing to coma and seizures. Blood levels of ammonia, uric acid, liver transaminases, and creatine phosphokinase may be elevated during acute episodes, and liver biopsy shows microvesicular steatosis. Autopsy shows fatty infiltration of the liver, renal tubules, and heart and skeletal muscle. The disorder was often misdiagnosed as Reye syndrome or sudden infant death syndrome, because the initial episode was fatal in about 25% of cases. Diagnosis through clinical symptoms is now rare as the disorder is readily identified through newborn screening by tandem mass spectrometry. Patients thus identified are typically well, though at risk for hypoglycemia with intercurrent illness, and fatalities are a rarity. A few enzyme-deficient individuals born prior to newborn screening have had their first presentation in adolescence or adult life and some have remained asymptomatic.

Analysis of serum acylcarnitines by tandem mass spectrometry shows elevations of C8, C8:1, and C10:1 esters even between episodes. The same abnormalities are identified through newborn screening. The C6, C8, and C10 dicarboxylic aciduria that occurs during acute episodes often should raise suspicion of the disease and biochemical confirmation can be obtained by measurement hexanoylglycine and suberylglycine in urine. Phenyipropionylglycine in urine will be elevated if the gut has been colonized by adult-type flora, but can be missed by all but the most sensitive techniques. Free carnitine in serum is usually low. Enzyme deficiency can be shown in fibroblasts or leukocytes but molecular diagnosis is more readily available and often faster.

Treatment of acute episodes in medium-chain acyl-CoA dehydrogenase deficiency is primarily supportive and aimed at quickly reversing the catabolic state that is responsible for stimulating the pathways of lipolysis and fatty acid oxidation. Hypoglycemia may be corrected with bolus administration of intravenous dextrose. Continuous infusion of dextrose should then be given at a rate that maintains plasma glucose levels at, or slightly above, the normal range in order to stimulate insulin secretion and suppress adipose tissue lipolysis. Specific therapy for the mild hyperammonemia that may be present during acute illness has not usually been required. Cerebral edema has occurred during treatment in some patients with severe coma, possibly as a late reflection of acute brain injury from hypoglycemia, toxic effects of fatty acids, or ischemia. Recovery from the acute metabolic derangements associated with coma may require more than a few hours, but is usually complete within 12 to 24 hours except where serious injury to the brain has occurred. Long-term management consists of dietary therapy to prevent excessive periods of fasting that can lead to coma. Overnight fasting in infants should be limited to no more than 8 hours. A duration of 12-18 hours is probably safe in children >1 year of age. Home blood glucose monitoring is not useful because symptomatic illness can begin before hypoglycemia has occurred. Although it is reasonable to modestly reduce dietary fat, because this fuel cannot be used efficiently in medium-chain acyl-CoA dehydrogenase deficiency, patients appear to tolerate normal diets without difficulty, and severe restriction of fat intake may be unnecessary. Formulas containing medium-chain triglycerides oil should be avoided. Although patients with medium-chain acyl-CoA dehydrogenase deficiency and other acyl-CoA oxidation defects have secondary carnitine deficiency, the use of carnitine supplementation in these disorders is controversial. Some investigators suggest 50 to 100 mg/day of oral carnitine but its utility is unproven.

The ACADM gene is on chromosome 1 (1p31), and MCAD deficiency is inherited as a recessive trait. The vast majority of patients with medium-chain acyl-CoA dehydrogenase deficiency have a single common missense mutation: an A-to-G transition at cDNA position 985, which changes a lysine residue to glutamate at amino acid 329 of the medium-chain acyl-CoA dehydrogenase precursor protein. The mutated amino acid is far removed from the catalytic site of the enzyme but appears to make the protein unstable by interfering with intramitochondrial folding and assembly of the nascent peptide. Preventing this misfolding offers an opportunity for development of new therapeutic agents for medium-chain acyl-CoA dehydrogenase deficiency. The A985G mutation accounts for approximately 90% of the mutant alleles in medium-chain acyl-CoA dehydrogenase deficiency. Approximately 70% of patients are homozygous for the A985G mutation. Most of the remaining patients are compound heterozygotes for the A985G allele in combination with 1 of several rarer mutations. Thus, only a few percent of medium-chain acyl-CoA dehydrogenase patients do not have at least one A985G allele. The unusually high frequency of a single common mutation has made molecular diagnosis especially valuable in medium-chain acyl-CoA dehydrogenase deficiency. As more information accumulates from patients identified through newborn screening, correlation of phenotype with genotype is becoming clearer. Patients with the common mutation accumulate the highest levels of metabolites in the newborn period and are probably at risk for more severe disease than are many other mutations.

Several chain length-specific NAD-dependent 3-hydroxyacyl-CoA dehydrogenases catalyze the oxidation of 3-hydroxyacyl-CoA esters to 3-ketoacyl esters. LCHAD acts on hydroxyacyl-CoAs longer than C₈. LCHAD and long-chain enoyl-CoA hydratase activities are carried on the α-subunit of the mitochondrial trifunctional protein (MTP or TFP), and long-chain 3-ketoacyl-CoA thiolase is carried on the β-subunit. LCHAD deficiency can exist alone, or together with deficiency of the other two enzymes.

Patients with a deficiency of LCHAD tend to fall into two clinical subclasses. One group presents primarily with symptoms of cardiomyopathy, myopathy, and hypoglycemia. Peripheral neuropathy and recurrent myoglobinuria may be present. These patients are deficient in all three enzymatic activities of the trifunctional protein. The other group, deficient only in LCHAD activity, has hepatocellular disease with hypoglycemia with or without pigmentary retinopathy. Cholestasis and fibrosis may also be present. Considerable overlap in these groups has been described, however, and LCHAD deficiency has also been reported in patients with recurrent Reye syndrome-like symptoms and in sudden infant death. Milder cases with adolescent onset of recurrent rhabdomyolysis have been reported. Fetal LCHAD deficiency frequently causes acute fatty liver or HELLP syndrome (hemolysis, elevated liver enzymes, and low platelets) in the (heterozygous) mother during pregnancy, especially when one or both mutant alleles in the fetus is E474Q.

Acylcarnitine analysis by tandem mass spectrometry is usually diagnostic including in the newborn period, and shows elevated saturated and unsaturated C16 and C18 hydroxyacylcarnitines. Organic acid analysis often shows elevated C6-14 3-hydroxydicarboxylic acids, but the same abnormalities have been seen in patients with respiratory chain defects and glycogenoses, and are not specific. The enzyme defect can be demonstrated in fibroblasts and leukocytes and, for prenatal diagnosis, in amniocytes.

Therapeutic options and controversies parallel those for VLCAD deficiency. In addition docosahexaenoic acid, a polyunsaturated C₂₀ acid, has been proposed to slow the development of retinitis but remains under investigation.

Long-chain hydroxyacyl-CoA dehydrogenase (LCHAD) deficiency, whether isolated or part of trifunctional protein deficiency, is inherited as an autosomal recessive trait, as the genes for both subunits (HADHA and HADHB) are located on chromosome 2 (2p24.1-23.3). Several disease-causing mutations have been identified, and most affect the α-subunit. One of these, E510Q (E474Q in the mature subunit), accounts for nearly 90% of mutant alleles in patients of European extraction with isolated LCHAD deficiency. Defects in the β-subunit tend to destabilize the trifunctional protein resulting in the multiple enzymatic deficiencies seen in some patients. Prenatal diagnosis can be made by enzyme assay in amniocytes or chorionic villus samples or, when appropriate, by mutation analysis, and on occasion will be indicated to avoid the complications of pregnancy.

Electrons from the acyl-CoA dehydrogenases involved in mitochondrial fatty acid and amino acid oxidation are transferred from their FAD coenzymes to coenzyme Q in the respiratory chain via electron transfer flavoprotein (ETF) and ETF:ubiquinone oxidoreductase (ETF:QO). Defects in ETF and ETF:QO cause multiple acyl-CoA dehdyrogenase deficiency (MADD), often called glutaric acidemia type II because of one of the characteristic metabolites that accumulates.

Glutaric acidemia type II was first described in 1976 in a baby who died at 3 days of age with severe hypoglycemia, metabolic acidosis, and the smell of sweaty feet, and many additional patients have since been described. Clinical manifestations are extremely heterogeneous. A neonatal form can be seen with severe hypotonia, dysmorphic features, and cystic kidneys. These infants also exhibit metabolic acidosis and hypoglycemia. Milder variants are common, presenting with non-specific neurological signs, lipid storage myopathy, fasting hypoketotic hypoglycemia, and/or intermittent acidosis. In some patients, only fasting hypoketotic hypoglycemia and/or intermittent acidosis is seen and can be of late onset. In these cases, the organic acid profile in times of illness is usually dominated by ethylmalonic and adipic acids, leading to the alternate name of ethylmalonic-adipic aciduria for this disorder. Structural brain abnormalities are common including agenesis of the cerebellar vermis, hypoplastic temporal lobes, and focal dysplasia of cerebral cortex. Neuronal migration abnormalities may be present. Riboflavin responsive mutations in the ETFDH gene have been reported.

Organic acid analysis usually shows increased ethylmalonic, glutaric, 2-hydroxyglutaric, and 3-hydroxyisovaleric acids, together with C₆, C₈, and C₁₀ dicarboxylic acids and isovalerylglycine, and acylcarnitine analysis by tandem mass spectrometry shows glutarylcarnitine, isovalerylcarnitine, and straight-chain esters of chain length C4, C8, C10, C10:1, and C12. Serum carnitine is usually low, and serum sarcosine is often increased in patients with mild disease. Enzyme or immunoblot analyses, if necessary, will show that some patients are deficient in ETF, and that others are deficient in ETF:QO. Molecular testing is typically more readily available.

Patients with complete defects often die during the first weeks of life, usually of conduction defects or arrhythmias, but those with incomplete defects can survive well into adult life. As in other fatty acid oxidation disorders, treatment relies on the avoidance of fasting, sometimes with continuous intragastic feeding, and carnitine to replenish lost stores. Riboflavin is usually given, and appears to have helped some patients. Carnitine supplementation (100 mg/kg/day) will increase metabolite excretion and should be used.

Electron transfer flavoprotein (ETF) and electron transfer flavoprotein:ubiquinone oxidoreductase (ETF:QO) deficiency are both inherited as autosomal recessive traits, and the genes encoding ETF:QO and the α- and β-subunits of ETF have been mapped to chromosome 4 (4q32>ter), 15 (15q23-25), and 19 (19q13.3), respectively. Disease-causing mutations have been identified in all three genes, but only in the ETFA gene is there is a common mutant allele (T266M). Severe forms of the disease have been diagnosed in utero by demonstrating increased amounts of glutaric acid in amniotic fluid, and in some cases renal cysts have been seen in the fetus on ultrasound examination.

### Mitochondrial Respiratory Chain Deficiencies

Mitochondria play an important role in cellular energy metabolism, free radical generation, and apoptosis. Mitochondria are the main source of reactive oxygen species (ROS) generation in cells, and mitochondrial dysfunction can contribute to cell damage through increased generation of ROS. Electrons channeling through the electron transfer chain (ETC) can escape the electron transfer complexes and react with molecular oxygen to form superoxide radicals (O⁻²). Impaired electron transfer at complexes I and III have been shown to increase intracellular levels of O⁻², H₂O₂, and ROS. ROS subsequently is important in the regulation many cellular functions, acting to activate specific transcription factors. However, excessive generation of ROS can also cause oxidative damage to the mitochondria and cells. It has been demonstrated that the rate of production of O⁻² and H₂O₂ and ROS in mitochondria increases with aging in various tissues in humans and animals.

Agents (e.g., compounds) useful in the treatment methods provided herein are mitochondria-targeting electron, radical and reactive oxygen species (ROS) scavengers, comprising a mitochondria targeting moiety attached covalently to an electron, radical and ROS scavenging moiety. An “electron, radical and ROS scavenger” is a compound or moiety that captures electrons, released, for example, from the electron-transfer chain (ETC) in the inner mitochondrial membrane, or a compound that reacts covalently and irreversibly with other compounds containing unpaired electrons, or a compound that reacts with ROS such as superoxide radical anion, hydroxyl radicals, or reactive nitrogen species (RNS). Non-limiting examples of electron, radical and ROS scavengers or moieties include compounds with a N—O., N—OH, or N═O-containing moiety, e.g., a nitroxide-containing moiety, that can be attached by any useful chemistry to a mitochondria-targeting group, such as a specific peptide or alkene peptide isostere sequence, for example the alkene peptide isostere sequence used in XJB-5-131, and retains its scavenging capacity, e.g. in mitochondria. A “mitochondria targeting moiety” is a moiety (that is, a part of a molecule) that partitions specifically to mitochondria. In one aspect, a mitochondria targeting moiety is a membrane active peptide fragment derived from an antibiotic molecule that acts by targeting a bacterial cell wall. Examples of such antibiotics include: bacitracins, gramicidins, valinomycins, enniatins, alamethicins, beauvericin, serratomolide, sporidesmolide, tyrocidins, polymyxins, monamycins, and lissoclinum peptides.

The membrane-active peptide fragment derived from an antibiotic may comprise a complete antibiotic polypeptide, or a portion thereof having mitochondria-targeting abilities, which is readily determined, for example, by cellular partitioning experiments using radiolabeled peptides. Examples of useful gramicidin-derived membrane active peptide fragments are the Leu-DPhe-Pro-Val-Orn and DPhe-Pro-Val-Orn-Leu hemigramicidin S fragments. As gramicidin S is cyclic, any hemigramicidin 5-mer is expected to be useful as a membrane active peptide fragment, including Leu-DPhe-Pro-Val-Orn, DPhe-Pro-Val-Orn-Leu, Pro-Val-Orn-Leu-DPhe, Val-Orn-Leu-DPhe-Pro and Orn-Leu-DPhe-Pro-Val (from gramicidin S). Any larger or smaller fragment of gramicidin, or even larger fragments containing repeated gramicidin sequences (e.g., Leu-DPhe-Pro-Val-Orn-Leu-DPhe-Pro-Val-Orn-Leu-DPhe-Pro) are expected to be useful for membrane targeting, and can readily tested for such activity. In one aspect, the gramicidin S-derived peptide comprises a β-turn, which appears to confer to the peptide a high affinity for mitochondrial membranes. Derivatives of gramicidin, or other antibiotic fragments, include isosteres (molecules or ions with the same number of atoms and the same number of valence electrons—as a result, they can exhibit similar pharmacokinetic and pharmacodynamic properties), such as (E)-alkene isosteres (see, United States Patent Publication Nos. 2007/0161573 and 2007/0161544, incorporated herein by reference in their entirety, for exemplary synthesis methods). As with gramicidin S, the structure (amino acid sequence) of bacitracins, other gramicidins, valinomycins, enniatins, alamethicins, beauvericin, serratomolide, sporidesmolide, tyrocidins, polymyxins, monamycins, and lissoclinum peptides are all broadly-known, and fragments of these can be readily prepared and their membrane-targeting abilities can easily be confirmed by a person of ordinary skill in the art.

In another aspect, peptide isosteres, e.g., amide bond replacements of a portion of gramicidin S are employed as mitochondria-targeting groups. Among the suitable peptide isosteres are trisubstituted (E)-alkene peptide isosteres and cyclopropane peptide isosteres, as well as all imine addition products of hydro- or carbometalated internal and terminal alkynes for the synthesis of d-i and trisubstituted (E) alkene and cyclopropane peptide isosteres (See Wipf et al. Imine additions of internal alkynes for the synthesis of trisubstituted (E)-alkene and cyclopropane isosteres, Adv Synth Catal. 2005, 347:1605-1613). These peptide mimetics have been found to act as β turn promoters (See Wipf et al. Convergent Approach to (E)-Alkene and Cyclopropane Peptide Isosteres, Org Lett. 2005, 7(1): 103-106).

In one aspect, the mitochondria-targeting moiety is a membrane-active fragment of gramicidin S or an E-alkylene isostere thereof conjugated to a cargo, wherein the fragment of gramicidin S or E-alkylene isostere thereof has the sequence of one of: Leu-^{D}Phe-Pro-Val-Orn, Leu-(E)-^{D}Phe-Pro-Val-Orn, or acylated derivatives thereof, in which pendant amines are acylated or modified with an amine protecting group as indicated herein.

In another aspect, the compound has the structure:

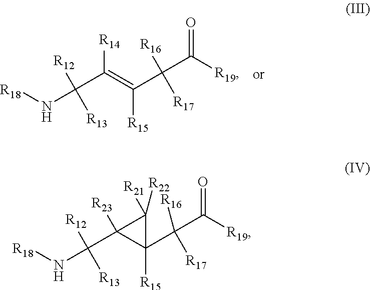

wherein R₁₂, R₁₃, R₁₆, and R₁₇ are independently hydrogen, hydroxyl, halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, for example, and without limitation, R₁₂, R₁₃, R₁₆, and R₁₇ are independently methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl, or hydroxyphenyl;
R₁₅ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted; R₁₈ is —C(O)—R₂₄. —C(O)O—R₂₄, or —P(O)—(R₂₄)₂, wherein R₂₄ is C₁-C₆ straight or branched-chain alkyl or a C₁-C₆ straight or branched-chain alkyl optionally comprising one or more (C₆H₅) groups that are independently unsubstituted, or methyl-, ethyl-, hydroxyl-, halo-substituted or fluoro-substituted, for example and without limitation, R₁₈ is Ac (Acetyl, R=—C(O)—CH₃), Boc (R=—C(O)O-tert-butyl), Cbz (R=—C(O)O-benzyl (Bn)), or a diphenylphosphate group;
R₁₉ is —NH—R₂₀, —O—R₂₀ or —CH₂—R₂₀, where R₂₀ is an electron, radical and ROS scavenging moiety, such as an N—O., N—OH, or N═O-containing moiety, e.g., a nitroxide-containing moiety, that can be conjugated by any useful chemistry to a mitochondria-targeting group, such as the alkene peptide isostere sequence in XJB-5-131 (see, generally United States Patent Publication No. 20140018317, incorporated herein by reference in its entirety, providing a list in Table 1 thereof of exemplary N—O., N—OH, or N═O-containing moieties); R₁₄ is a halo, a C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; and
R₂₁, R₂₂, and R₂₃ are independently H or halogens (See, e.g., International Patent Publication Nos. WO 2010/009405 and WO 2012/112851, incorporated herein by reference in their entirety).

In another aspect, the compound has the structure:

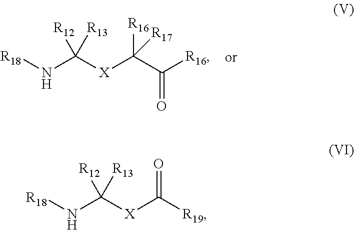

wherein X is

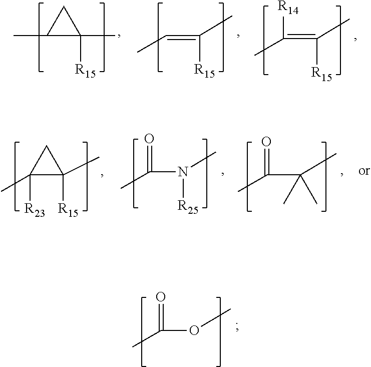

R₁₂, R₁₃, R₁₆, R₁₇, and R₂₅ are each independently hydrogen, halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted;
R₁₉ is —NH—R₂₀, —O—R₂₀ or —CH₂—R₂₀, where R₂₀ is an electron, radical and ROS scavenging moiety, such as an N—O., N—OH, or N═O-containing moiety, e.g., a nitroxide-containing moiety, that can be conjugated by any useful chemistry to a mitochondria-targeting group, such as the alkene peptide isostere sequence in XJB-5-131; and
R₁₈ is —C(O)—R₂₄, —C(O)O—R₂₄, or —P(O)—(R₂₄)₂, wherein R₂₄ is C₁-C₆ straight or branched-chain alkyl or a C₁-C₆ straight or branched-chain alkyl optionally comprising one or more (C₆H₅) groups that are independently unsubstituted, or methyl-, ethyl-, hydroxyl-, halo-substituted or fluoro-substituted, for example and without limitation, R₁₈ is Ac (Acetyl, R=—C(O)—CH₃), Boc (R=—C(O)O-tert-butyl), Cbz (R=—C(O)O-benzyl (Bn)), or a diphenylphosphate group.

Non-limiting examples of compounds according to (V) include:

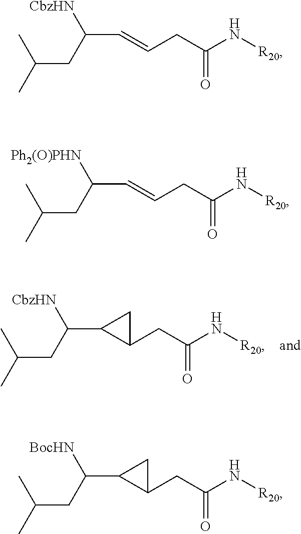

wherein R₂₀ is an electron, radical, or ROS-scavenging moiety, such as an N—O., N—OH, or N═O-containing moiety, e.g., a nitroxide-containing moiety, that can be covalently attached by any useful chemistry to a mitochondria-targeting group, such as the alkene peptide isostere sequence in XJB-5-131.

In one aspect, the compound has the structure:

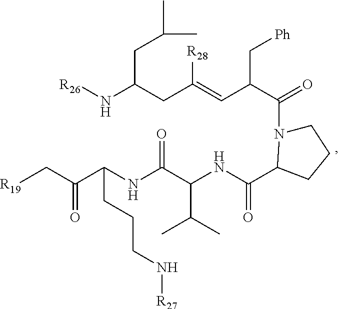

e.g.,

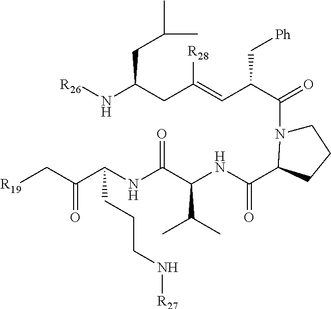

wherein R₁₉ is —NH—R₂₀, —O—R₂₀ or —CH₂—R₂₀, where R₂₀ is an electron, radical, or ROS-scavenging agent, such as an N—O., N—OH, or N═O-containing moiety, e.g., a nitroxide-containing moiety, that can be covalently attached by any useful chemistry to a mitochondria-targeting group, such as the alkene peptide isostere sequence in XJB-5-131; R₂₆ and R₂₇, independently are an amine protecting group or acylated. In one aspect, R₂₆ and R₂₇ are protecting groups independently selected from the group consisting of: 9-fluorenylmethyloxy carbonyl (Fmoc), t-butyloxycarbonyl (Boc), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), 1-(4,4-dimethyl-2,6-dioxacyclohexylidene)ethyl (Dde), diathiasuccinoyl (Dts), benzothiazole-2-sulfonyl (Bts), dimethoxytrityl (DMT) and monomethoxytrityl (MMT), and R₂₈ is H or methyl. In one aspect, R₂₆ is Boc and R₂₇ is Cbz. Ph is phenyl.

In another aspect, the compound has the structure:

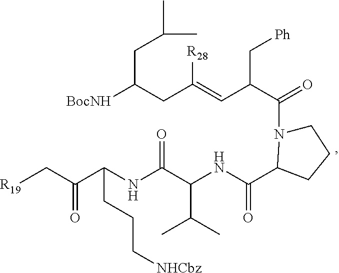

e.g.,

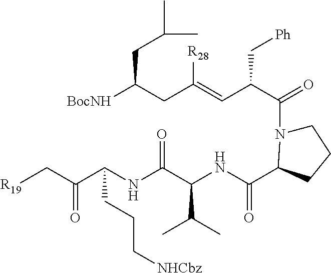

wherein R₁₉ is —NH—R₂₀, —O—R₂₀ or —CH₂—R₂₀, where R₂₀ is an electron, radical, or ROS-scavenging agent, such as an N—O., N—OH, or N═O-containing moiety, e.g., a nitroxide-containing moiety, that can be covalently attached by any useful chemistry to a mitochondria-targeting group, such as the alkene peptide isostere sequence in XJB-5-131.

In another aspect, the compound has the structure:

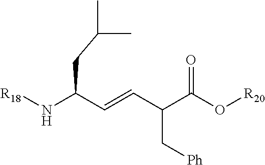

wherein R₂₀ is an electron, radical, or ROS-scavenging agent, such as an N—O., N—OH, or N═O-containing moiety, e.g., a nitroxide-containing moiety, that can be covalently attached by any useful chemistry to a mitochondria-targeting group, such as shown for XJB-5-131, and R₁₈ is an amine protecting group or acylated as described above for R₁₈ and R₁₉. In one aspect, R₁₈ is Boc or Cbz and R₂₀ is TEMPO or a nitroxide-containing group and in another R₁₈ is Boc and R₂₀ is TEMPO.

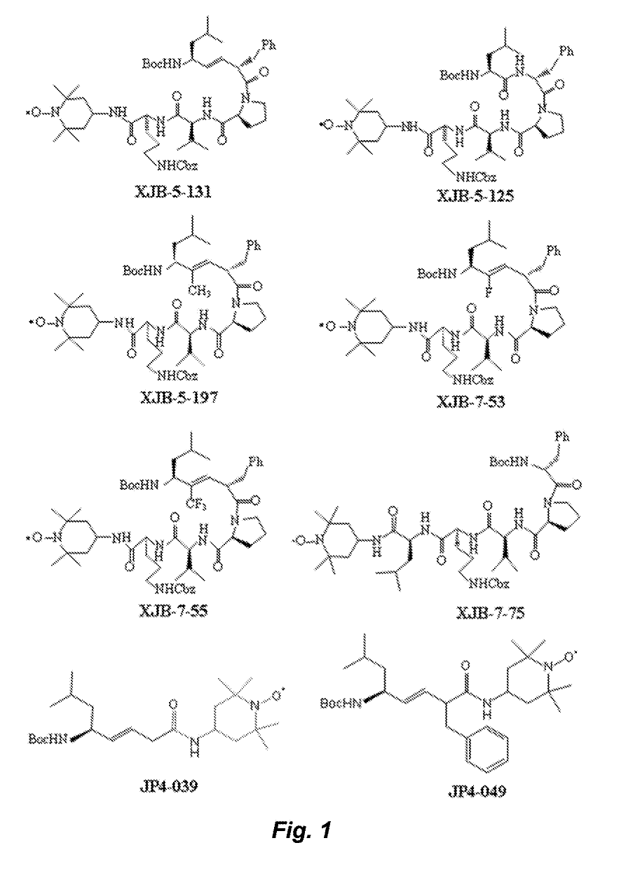

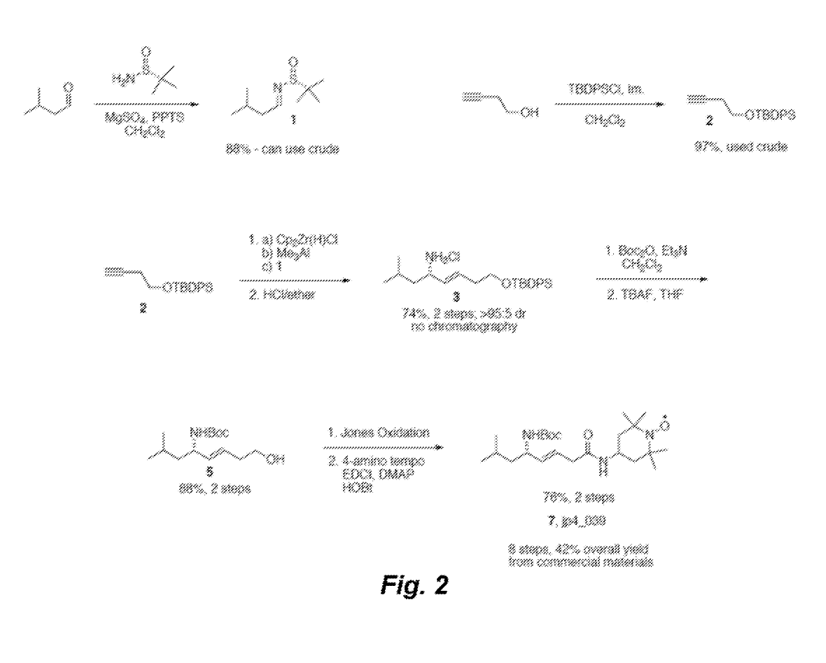

In one aspect, the compound is one or more of: XJB-5-125, XJB-5-131, XJB-5-197, XJB-7-53, XJB-7-SS. XJB-7-75, JP4-039 and JP4-049 (FIG. 1). Synthesis of those compounds are described in United States Patent Publication No. 20140018317, and in U.S. Pat. No. 7,718,608, incorporated herein by reference in its entirety, and elsewhere. FIG. 2 shows a synthesis scheme for JP4-039.

As used herein, unless indicated otherwise, for instance in a structure, all compounds and/or structures described herein comprise all possible stereoisomers, individually or mixtures thereof.

The following are exemplary definitions of various structural elements described herein. As used herein, “alkyl” refers to straight, branched chain, or cyclic hydrocarbon groups including from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. “Substituted alkyl” refers to alkyl substituted at 1 or more, e.g., 1, 2, 3, 4, 5, or even 6 positions, which substituents are attached at any available atom to produce a stable compound, with substitution as described herein. “Optionally substituted alkyl” refers to alkyl or substituted alkyl. “Halogen,” “halide,” and “halo” refers to —F, —CI, —Br, and/or —I. “Alkylene” and “substituted alkylene” refer to divalent alkyl and divalent substituted alkyl, respectively, including, without limitation, ethylene (—CH₂—CH₂—). “Optionally substituted alkylene” refers to alkylene or substituted alkylene.

“Alkene or alkenyl” refers to straight, branched chain, or cyclic hydrocarbyl groups including from 2 to about 20 carbon atoms, such as, without limitation C₁₋₃, C₁₋₆, C₁₋₁₀ groups having one or more, e.g., 1, 2, 3, 4, or 5, carbon-to-carbon double bonds. “Substituted alkene” refers to alkene substituted at 1 or more, e.g., 1, 2, 3, 4, or 5 positions, which substituents are attached at any available atom to produce a stable compound, with substitution as described herein. “Optionally substituted alkene” refers to alkene or substituted alkene. Likewise, “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. The term “alkynylene” refers to divalent alkyne. Examples of alkynylene include without limitation, ethynylene, propynylene. “Substituted alkynylene” refers to divalent substituted alkyne.

The term “alkoxy” refers to an —O-alkyl group having the indicated number of carbon atoms. For example, a (C₁-C₆)alkoxy group includes —O-methyl (methoxy), —O-ethyl (ethoxy), —O-propyl (propoxy), —O-isopropyl (isopropoxy), —O-butyl (butoxy), —O-sec-butyl (sec-butoxy), —O-tert-butyl (tert-butoxy), —O-pentyl (pentoxy), —O-isopentyl (isopentoxy), —O-neopentyl (neopentoxy), —O-hexyl (hexyloxy), —O-isohexyl (isohexyloxy), and —O-neohexyl (neohexyloxy). “Hydroxyalkyl” refers to a (C₁-C₁₀)alkyl group wherein one or more of the alkyl group's hydrogen atoms is replaced with an —OH group. Examples of hydroxyalkyl groups include, but are not limited to, —CH₂OH, —CH₂CH₂OH, —CH₂CH₂OCH₂OH, —CH₂CH₂CH₂CH₂OH, —CH₂CH₂CH₂CH₂CH₂OH, —CH₂CH₂CH₂CH₂CH₂CH₂OH, and branched versions thereof. The term “ether” or “oxygen ether” refers to (C₁-C₁₀)alkyl group wherein one or more of the alkyl group's carbon atoms is replaced with an —O— group. The term ether includes —CH₂—(OCH₂—CH₂)_{q}OP₁ compounds where P₁ is a protecting group, —H, or a (C₁-C₁₀)alkyl. Exemplary ethers include polyethylene glycol, diethylether, methylhexyl ether and the like.

“Aryl,” alone or in combination refers to an aromatic monocyclic or bicyclic ring system such as phenyl or naphthyl. “Aryl” also includes aromatic ring systems that are optionally fused with a cycloalkyl ring. A “substituted aryl” is an aryl that is independently substituted with one or more substituents attached at any available atom to produce a stable compound, wherein the substituents are as described herein. “Optionally substituted aryl” refers to aryl or substituted aryl. “Arylene” denotes divalent aryl, and “substituted arylene” refers to divalent substituted aryl. “Optionally substituted arylene” refers to arylene or substituted arylene.

“Heteroatom” refers to N, O, P and S. Compounds that contain N or S atoms can be optionally oxidized to the corresponding N-oxide, sulfoxide or sulfone compounds. “Hetero-substituted” refers to an organic compound in any embodiment described herein in which one or more carbon atoms are substituted with N, O, P or S.

“Substituted” or “substitution” refer to replacement of a hydrogen atom of a molecule or an R-group with one or more additional R-groups such as halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, nitro, sulfato or other R-groups.

“Cycloalkyl” refer to monocyclic, bicyclic, tricyclic, or polycyclic, 3- to 14-membered ring systems, which are either saturated, unsaturated or aromatic. The cycloalkyl group may be attached via any atom. Cycloalkyl also contemplates fused rings wherein the cycloalkyl is fused to an aryl or heteroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with 1, 2 or 3 substituents, attached at any available atom to produce a stable compound, wherein the substituents are as described herein.

“Carboxyl” or “carboxylic” refers to group having the indicated number of carbon atoms and terminating in a —C(O)OH group, thus having the structure —R—C(O)OH, where R is a divalent organic group that includes linear, branched, or cyclic hydrocarbons. Non-limiting examples of these include: C₁₋₈ carboxylic groups, such as ethanoic, propanoic, 2-methylpropanoic, butanoic, 2,2-dimethylpropanoic, pentanoic, etc.

“(C₃-C₈)aryl-(C₁-C₆)alkylene” refers to a divalent alkylene wherein one or more hydrogen atoms in the C₁-C₆ alkylene group is replaced by a (C₃-C₈)aryl group. Examples of (C₃-C₈)aryl-(C₁-C₆)alkylene groups include without limitation 1-phenylbutylene, phenyl-2-butylene, 1-phenyl-2-methylpropylene, phenylmethylene, phenylpropylene, and naphthylethylene. The term “(C₃-C₈)cycloalkyl-(C₁-C₆)alkylene” refers to a divalent alkylene wherein one or more hydrogen atoms in the C₁-C₆ alkylene group is replaced by a (C₃-C₈)cycloalkyl group. Examples of (C₃-C₈)cycloalkyl-(C₁-C₆)alkylene groups include without limitation 1-cyclopropylbutylene, cyclopropyl-2-butylene, cyclopentyl-1-phenyl-2-methylpropylene, cyclobutylmethylene and cyclohexylpropylene.

“Halo” refers to halogens, including F, Cl, Br, and I.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned herein are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely the salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like. The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen.

Pharmaceutically acceptable counterions include chloro, bromo, iodo, trifluoroacetate and acetate. The counterion of choice can be introduced using ion exchange resins.

“Pharmaceutically acceptable esters” includes those derived from compounds described herein that are modified to include a carboxyl group. An in viva hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C₁₋₄ alkyl, or C₁₋₄ alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and valyl.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in viva biotransformation product of the derivative is the active drug as defined in the compounds described herein. Prodrugs preferably have excellent aqueous solubility, increased bioavailability and are readily metabolized into the active inhibitors in vive. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art.

The term “prodrug” also is intended to include any covalently bonded carriers that release an active parent drug of the present invention in vive when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

As used herein, unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

As used herein, a ring structure showing a bond/group that is not attached to any single carbon atom, for example and without limitation, depicted as

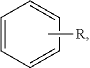

can be substituted at any position with one or more groups designated “R”, and, unless indicated otherwise, each instance of R on the ring can be (independently) the same or different from other R moieties on the ring. Thus, if R is H, the group contains nothing but H groups. If R is “halo”, it is a single halo (e.g., F, Cl, Br and I) group. If R is one or more independently of halo and CN, the ring may comprise one, two, three, four, halo or CN groups, such as, for example and without limitation: 2, 3, 4, or 5 chloro; 2, 3, 4, or 5 bromo; 2, 3- or 3,4- or 4,5- or 2,4-dichloro; 3-bromo-4-chloro; 3-bromo-4-cyano, and any other possible permutation of the listed groups.

Protected derivatives of the disclosed compounds also are contemplated. Many suitable protecting groups for use with the disclosed compounds are broadly-known in the art. In general, protecting groups are removed under conditions which will not affect the remaining portion of the molecule. These methods are well known in the art and include acid hydrolysis, hydrogenolysis and the like. One method involves the removal of an ester, such as cleavage of a phosphonate ester using Lewis acidic conditions, such as in TMS-Br mediated ester cleavage to yield the free phosphonate. A second method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with any of the large number of broadly-available publications. When an amine is deprotected, the resulting salt can readily be neutralized to yield the free amine. Similarly, when an acid moiety, such as a phosphonic acid moiety is unveiled, the compound may be isolated as the acid compound or as a salt thereof.

According to one aspect, amine side chains are protected using protective groups, for example and without limitation by acylation (See, e.g., U.S. Pat. Nos. 7,528,174; 7,718,603; and 9,006,186, and International Patent Publication Nos. WO 2010/009405 and WO 2012/112851, each of which are incorporated herein by reference in their entirety). Protecting groups are known in the art and include, without limitation: 9-fluorenylmethyloxy carbonyl (Fmoc), t-butyloxycarbonyl (Boc), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), 1-(4,4-dimethyl-2,6-dioxacyclohexylidene)ethyl (Dde), diathiasuccinoyl (Dts), benzothiazole-2-sulfonyl (Bts), dimethoxytrityl (DMT) and monomethoxytrityl (MMT) groups. A protecting group also includes acyl groups, such as acetyl groups, for example, as described.

The compounds typically are administered in an amount and dosage regimen to treat a fatty acid oxidation metabolic condition, such as inborn errors of fatty acid oxidation or oxidative phosphorylation. In various aspects of the invention, the mitochondria-targeting electron, radical, or ROS-scavenging compounds described herein are administered in any manner that is effective to treat, mitigate or prevent any fatty acid oxidation metabolic condition, such as inborn errors of fatty acid oxidation or oxidative phosphorylation, as described above, and/or hypoglycemia, rhabdomyolysis, or cardiomyopathy, e.g., as caused by a fatty acid oxidation metabolic condition, such as inborn errors of fatty acid oxidation or oxidative phosphorylation. Examples of delivery routes include, without limitation: topical, for example, epicutaneous, inhalational, enema, ocular, otic and intranasal delivery; enteral, for example, orally, by gastric feeding tube and rectally; and parenteral, such as, intravenous, intraarterial, intramuscular, intracardiac, subcutaneous, intraosseous, intradermal, intrathecal, intraperitoneal, transdermal, iontophoretic, transmucosal, epidural and intravitreal, with oral, intravenous, intramuscular and transdermal approaches being preferred in many instances.

As indicated above, and in the examples, depending upon the dosage of the compounds, the compounds can exhibit electron, radical, or ROS-scavenging activities, e.g., from 0.1 nM to 100 μM or 0.1 mg to 2 mg JP4-039 per Kg body weight of a patient, or doses of agents other than JP4-039 equivalent to a range of from 0.1 nM to 100 μM in a patient, or 0.1 mg to 2 mg of JP4-039 per Kg body weight, e.g., as described in the examples below. Therefore, an “effective amount” of the compound or composition described herein is an amount effective in a dosage regimen (amount of the compound and timing of delivery), to achieve a desired end-point, such as maintaining concentrations at a site of treatment within a range effective to achieve a suitable outcome. Suitable outcomes include improvement of one or more symptoms of a fatty acid oxidation or oxidative phosphorylation metabolic condition, such as hypoglycemia, rhabdomyolysis, lactic acidosis, hyperammonemia, skeletal myopathy, hypotonia, fatiguabilty, or cardiomyopathy.

The compounds may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include: intravenous, intramuscular, or intraperitoneal solutions, oral tablets or liquids, topical ointments or creams and transdermal devices (e.g., patches). In one embodiment, the compound is a sterile solution comprising the active ingredient (drug, or compound), and a solvent, such as water, saline, lactated Ringer's solution, or phosphate-buffered saline (PBS). Additional excipients, such as polyethylene glycol, emulsifiers, salts and buffers may be included in the solution.

In one aspect, the dosage form is a transdermal device, or “patch”. The general structure of a transdermal patch is broadly known in the pharmaceutical arts. A typical patch includes, without limitation: a delivery reservoir for containing and delivering a drug product to a subject, an occlusive backing to which the reservoir is attached on a proximal side (toward the intended subject's skin) of the backing and extending beyond, typically completely surrounding the reservoir, and an adhesive on the proximal side of the backing, surrounding the reservoir, typically completely, for adhering the patch to the skin of a patient. The reservoir typically comprises a matrix formed from a non-woven (e.g., a gauze) or a hydrogel, such as a polyvinylpyrrolidone (PVP) or polyvinyl acetate (PVA), as are broadly known. The reservoir typically comprises the active ingredient absorbed into or adsorbed onto the reservoir matrix, and skin permeation enhancers. The choice of permeation enhancers typically depends on empirical studies. Certain formulations that may be useful as permeation enhancers include, without limitation: DMSO; 95% Propylene Glycol+5% Linoleic Acid; and 50% EtOH+40% HSO+5% Propylene Glycol+5% Brij30.

Therapeutic/pharmaceutical compositions are prepared in accordance with acceptable pharmaceutical procedures, such as described in Remington: The Science and Practice of Pharmacy. 21st edition, ed. Paul Beringer et al., Lippincott, Williams & Wilkins, Baltimore, Md. Easton, Pa. (2005) (see, e.g., Chapters 37, 39, 41, 42 and 45 for examples of powder, liquid, parenteral, intravenous and oral solid formulations and methods of making such formulations).

### Example 1

The mitochondrial respiratory chain requires hundreds of proteins encoded by the nuclear and mitochondrial genomes for normal assembly and function. Mutations in the genes for many of these proteins lead to a wide range of clinical phenotypes related to energy deficiency. Mutations in the mitochondrial DNA introduce an additional variable of clinical heterogeneity. Cells have many mitochondria, and each mitochondrion has multiple copies of the mitochondrial chromosome. Distribution of both is random in dividing cells and mitochondria. Thus a cell can contain two populations of mitochondria, normal and mutant, in varying ratios, a genetic situation known as heteroplasmy. The extreme phenotypic heterogeneity of mitochondrial respiratory chain deficiency and variable degrees of heteroplasmy in different tissues for mitochondrial DNA (mtDNA) mutations pose a practical challenge in recognizing these conditions. Estimates of the prevalence of mitochondrial disease vary among reports, and may be as high is 1:5,000 individuals.

Increased mitochondrial DNA (mtDNA) damage (resulting in inactivation of ETC proteins) is not only seen in aging, but also in a variety of disease states including neurodegenerative and neuromuscular disorders such as MELAS (Mitochondrial encephalomyopathy lactic acidosis), LHON (Leber Hereditary Optic Neuropathy), MERRF (myoclonic epilepsy with ragged red fibers), KSS (Keams-Sayre Syndrome), Parkinson disease (PD), Alzheimer disease (AD), and Huntington disease (HD). In addition, tumor development is often associated with mutations in and altered expression of the mtDNA-encoded subunits of complexes I, III, IV, and V. The location and type of oxidation-induced mtDNA mutations are often disease and tissue specific. MtDNA is usually located near the inner mitochondrial membrane, which is also the main location of ROS generation.

Failure of superoxide dismutase with aging, and the limited DNA repair mechanisms within mitochondria, results in 10-fold higher rate of mtDNA damage compared to nuclear DNA (nDNA). Impairment of ETC activity increases ROS generation, and the exponential increase in ROS production in the mitochondria of aged mammalian cells has been attributed to existence of a “vicious cycle”. The cycle starts with the generation of ROS by the ETC that then leads to mtDNA damage. Damage to mtDNA ETC protein subunit genes further impairs ETC, which in turn, generates more ROS and more mtDNA damage. The ‘vicious cycle’ hypothesis is based mostly on observations of increased ROS generation, mtDNA damage, mtDNA mutations and deletions, and phospholipids oxidation in animals and people with aging, but there is little experimental evidence to directly support it. Here, a “mild oxidative exposure” strategy is used to selectively induce mtDNA over nDNA damage. The results are consistent with the ‘vicious cycle’ hypothesis and an experimental system is described herein to study it.

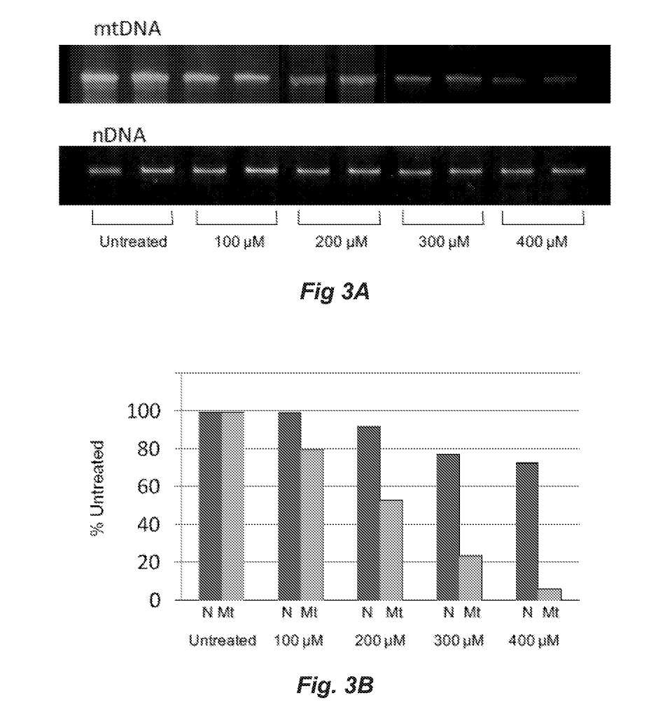

Selection of “mild oxidative exposure” conditions. In order to develop a model for mild oxidative exposure leading to mtDNA damage without affecting nDNA, cells were incubated with increasing concentrations of H₂O₂ for one hour and the total DNA was evaluated for evidence of oxidative damage by PCR. Reduction of the native 12 kb mtDNA and 13.4 kb nDNA fragments at each H₂O₂ concentration is shown in FIGS. 3A and 3B. Increasing concentrations of H₂O₂ exposure led to increased mtDNA damage but much lower levels of nDNA damage. Exposure to 200 μM H₂O₂ was selected for further experiments since it resulted in substantial damage to mtDNA (53.4% remaining normal fragments) but very little to nDNA (92.1% remaining normal fragments). Cells were then treated with 200 μM H₂O₂ for 1 hour/day for four days and returned to culture without further H₂O₂ exposure. This cell line is normally viable for many months. However, after the short term exposure to 200 μM H₂O₂, cells began to die 20 days after the last treatment.

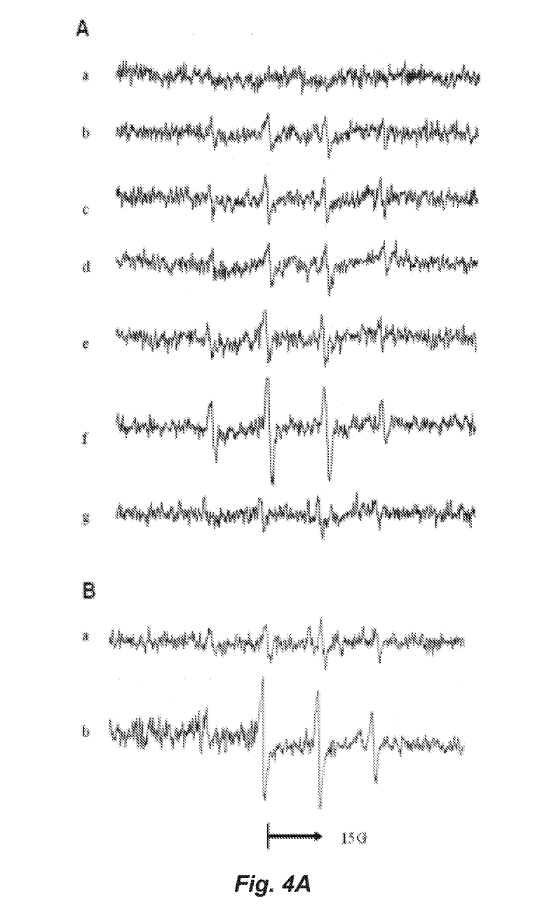

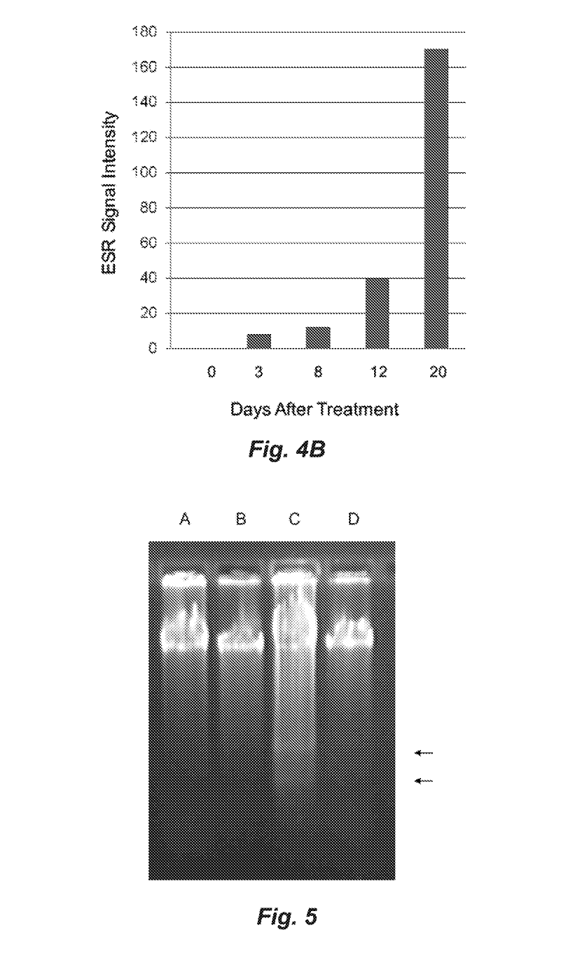

The level of late ROS production in cells exposed to 1202 was compared to untreated cells. Oxidation of DCFH by ROS was measured by ESR in treated and untreated cells (FIG. 4A(A)). Measurements were made in the presence of glutamate, malate, NADH, and succinate to ensure the presence of sufficient substrates for complex I, II and III of the electron transfer chain. No difference in the ESR signal intensity was detectable in untreated cells throughout the experiment (FIG. 4A(A)). In contrast, cells exposed to H₂O₂ exhibited an increase in ESR signal of 8% and 12% at 3 and 8 days, respectively, after exposure (FIGS. 4A(A.c), 4A(A.d) and 4B). This further increased by 30-50% on day 12 (FIGS. 4A(A.e) and 4B) and 165% by 20 days post exposure (FIGS. 4A(A.f) and 4B). ROS is generated during oxidative phosphorylation largely through the activities of ETC complexes I and III. To determine the possible site(s) of ROS generation in the H₂O₂ treated cells, NADH (the electron donor of complex I) and 2,3-dimethoxy-5-methyl-6-decyl-1,4-benzoquino (DBH2) (the electron donor of complex III) were separately incubated with mitochondria isolated from treated cells after 20 days, and ROS generation was measured by ESR. The ESR signal in the presence of DBH2 alone (FIG. 4A(B.a)), showed no difference compared to untreated cells (FIG. 4A(A.b)). However, in the presence of NADH, the ESR signal (FIG. 4A(B.b)) was 2.5 fold higher than in the presence of DBH2. These results suggest that the increased ROS is being generated through activity of complex I, but not complex III, and that complex I mtDNA sequences are more susceptible to internally generated ROS than complex III mtDNA sequences.

To measure late damage to nDNA following oxidative stress, total DNA was isolated and examined for appearance of fragments characteristic of apoptosis (FIG. 5). No damage was evident 5 days after the final H₂O₂ treatment (FIG. 5, lane B), but considerable accumulation of smaller fragments was present 15 days after H₂O₂ treatment (FIG. 5, lane C). Untreated cells showed no change initially (FIG. 5, lane A) or after 15 days of growth (FIG. 5, lane D). Thus, nDNA damage was occurring in an ongoing fashion even after removal of the external oxidative exposure. These results suggest that nDNA initially was not damaged by the relatively mild oxidative exposure. Rather, damage was by internally generated ROS during growth after removal of the external oxidative stress.

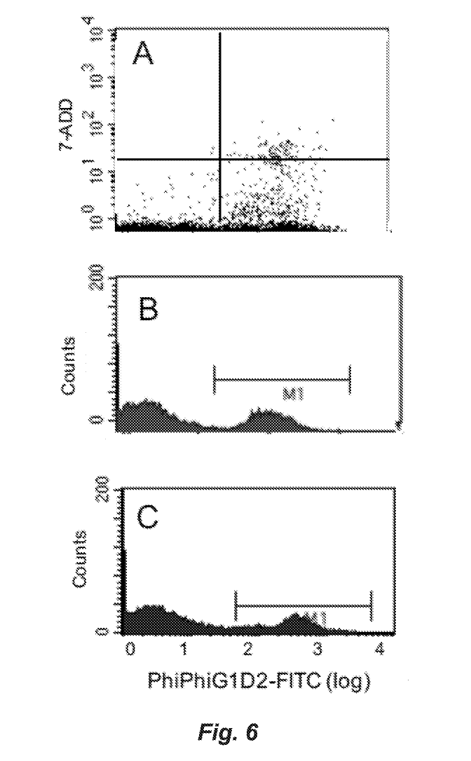

DNA damage is a strong inducer of apoptosis, which can be monitored by an increase in the activity of caspase 3. To test for apoptosis following H₂O₂ treatment, cells were incubated with the caspase 3 substrate PhiPhiLux-G1D2 3, 8, 12, and 20 days after the final day of exposure, and FACS sorted. Cells gating as 7 AAD(−) and PhiPhiLuxG1D2 (+) (FIG. 6(A)) represent apoptotic but not necrotic cells. By day 20 post exposure, the level of apoptosis in treated cells was 3-fold higher than in untreated cells (FIGS. 6(B and C), increasing concomitantly with ROS generation and nDNA fragmentation.

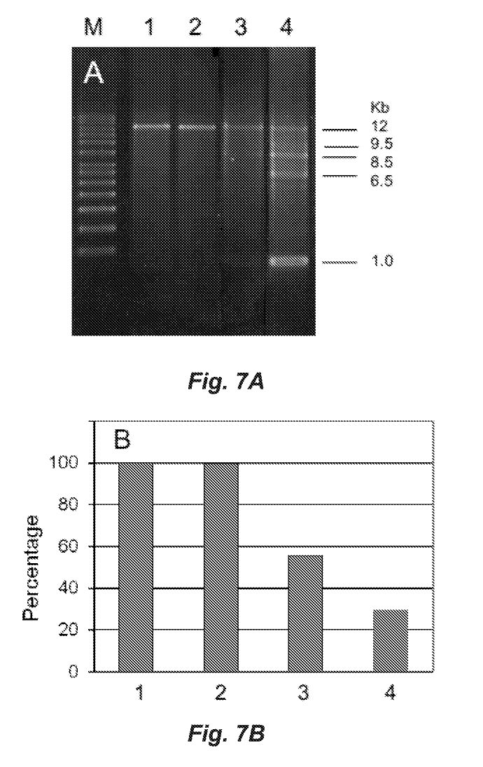

Late damage to mtDNA in cells exposed to oxidative stress was estimated by PCR (FIGS. 7A and 7B). Only the full length 12 kb mtDNA band was amplified from DNA of untreated cells at day 1 and day 20 (FIG. 7A, lanes 1 and 2). However, in the H₂O₂-treated cells, the 12 kb fragment was markedly reduced with the appearance of smaller molecular fragments as early as 3 days after the oxidative treatment (FIG. 7A, lane 3). This effect was exaggerated after 20 days, with the appearance of additional smaller-sized mtDNA fragments (FIG. 7A, lane 4), suggesting that mtDNA is being extensively damaged by internally generated ROS over time. FIG. 7B provides densitometer readings of the gel of FIG. 7A. Additional characterization of the 9.5 kb fragment by PCR revealed that the ND3-6 portion was absent while the DL and COX fragments were both present (data not shown).

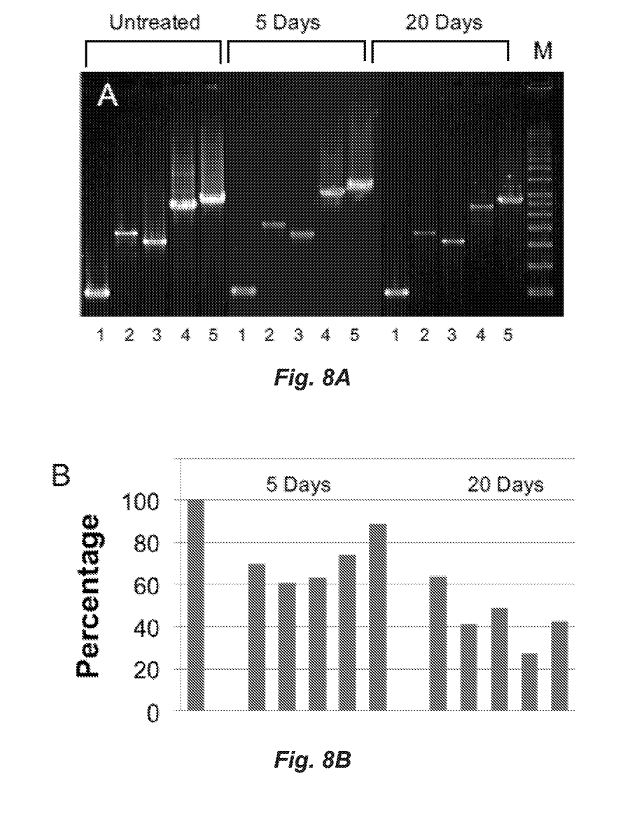

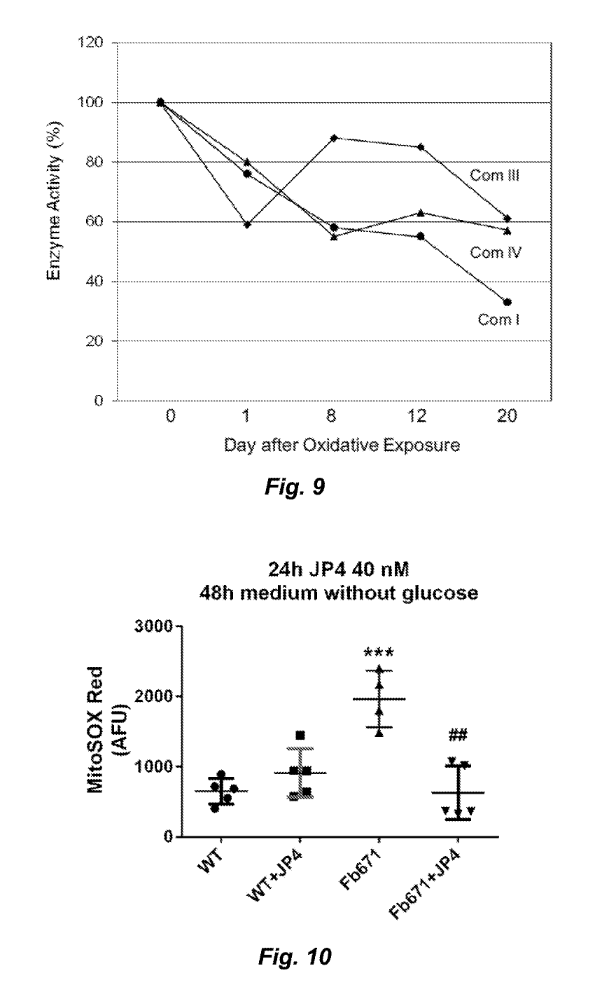

While damage to mtDNA first appeared at day 3 (FIGS. 9A and 9B), nDNA fragmentation was first identified at 15 days (FIG. 5), indicating greater susceptibility of the mtDNA to internally generated ROS. To identify differential susceptibility of regions on all mtDNA to oxidative damage, five pairs of mtDNA primers designed to amplify different portions of the mtDNA (Scheme 1 and Table 1). The PCR results are shown in FIG. 8A and the relative change in treated vs. untreated cells in FIG. 8B. All five mtDNA fragments (FIG. 5B) were significantly reduced in treated cells over time, but the reduction of each of the fragments varied. The Dloop fragment was least reduced, indicating a lower sensitivity of this region to oxidative stress, while the COX, ND1-2 and ND3-6 fragments were most affected.

The activities of ETC complexes 1, III, and IV were all reduced in cells following exposure to H₂O₂ (FIG. 9). It is likely that the reduction in enzyme activities three days after exposure is at least partially attributable to the external oxidative exposure. However, the progressive decrease in the activity of complexes I and IV as well as the rebound decrease in complex III is well beyond the window of initial damage and is consistent with new damage related to internally generated ROS. In addition, complex I was the most affected activity at day 20 (70% reduction), consistent with the findings demonstrated by ESR.

Treatment of Oxidative Stress with Next Generation Anti-Oxidants. A recently developed analog of the antibiotic gramicidin S, XJB-5-131 (XJB) targets mitochondria and provides mitoROS and electron scavenging capacity by virtue of its conjugation to a 4-amino-2,2,6,6-tetramethylpiperidinooxy (4-Amino-TEMPO) moiety. JP4-039, a mitochondrial-targeted TEMPOL, can be administered by intravenous, intraperitoneal, or swallowed route, and is associated with no acute toxicity. Reduction in cellular superoxide production is demonstrated in a cell line with a fatty acid oxidation defect (FIG. 10). It is thus concluded that treatment of inborn errors of fatty acid oxidation or oxidative phosphorylation with XJB-5-131, JP4-039, and other next generation mitochondrial targeted electron, radical, or ROS-scavenging agents will provide physiologic benefit and ameliorate disease.

### Example 2

Mitochondrial fatty acid n-oxidation (FAO) pathway spiral is an essential supplier of acetyl-CoA, the major source of reducing equivalents that drives ATP synthesis. Recessively inherited defects are known for most of the genes of FAO proteins, the most common being medium-chain acyl-CoA dehydrogenase (MCAD) and very long-chain acyl-CoA dehydrogenase (VLCAD) deficiencies. These are characterized by hypoglycemia, rhabdomyolysis, and cardiomyopathy. The pathophysiology of these abnormalities, however, is yet to be determined.

In this study, MCAD and VLCAD deficient fibroblasts were cultured in the absence of glucose for 48-72 h to shift to fatty acids as the energy source. Oxygen consumption rate (OCR) was monitored by a Seahorse XF analyzer. Mitochondrial mass was examined by MitoTracker Green and ROS generation by MitoSOX dye. Protein immunocontent was evaluated by western blotting.

Cell culture: Patient skin biopsies for fibroblast culture were performed on a clinical basis with written informed consent from patients and/or parents. Fibroblasts were cultured in Dulbecco's Modified Eagle Medium (DMEM) supplemented with 10% fetal bovine serum and 100 μg/ml penicillin/streptomycin, 4.5 g/l glucose, 4 mM glutamine and 2 mM pyruvate.

Oxygen consumption assay: Fibroblasts controls and from patients with VLCAD deficiency were cultivated in normal media or in the absence of glucose for 72 hours. The oxygen consumption was monitored by a Seahorse XFe96 Extracellular Flux Analyzer (Seahorse Bioscience, Billerica, Mass.).

Mitochondrial mass: Control and patient fibroblasts with VLCAD deficiency were cultivated in normal media or without glucose for 48 hours, then incubated for 25 min with MitoTracker Green FM (Life Technologies). Fluorescence was evaluated by flow cytometry.

ROS generation: control and patient fibroblasts were cultivated in normal media or without glucose for 48 hours, then incubated for 15 min with MitoSox Red (Life Technologies). Fluorescence was evaluated by flow cytometry.

### Results

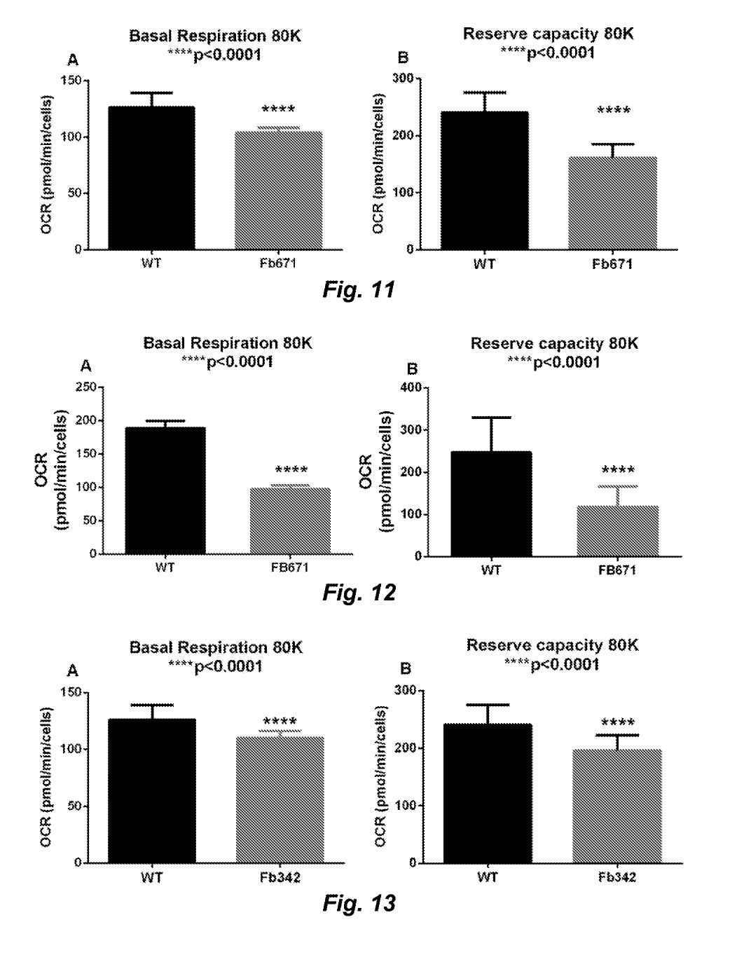

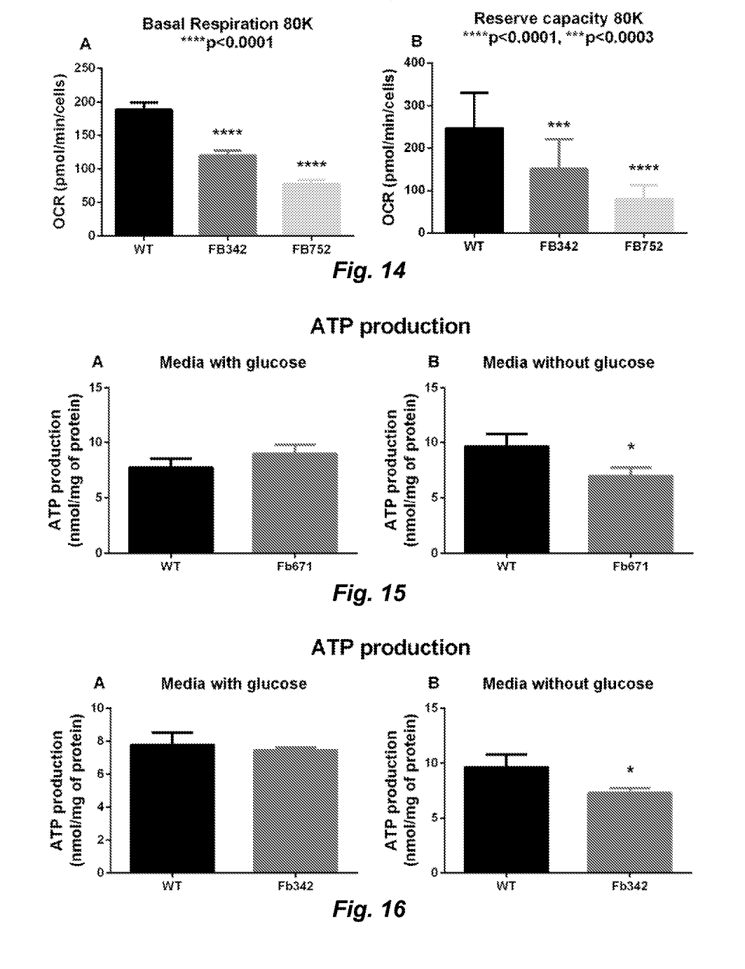

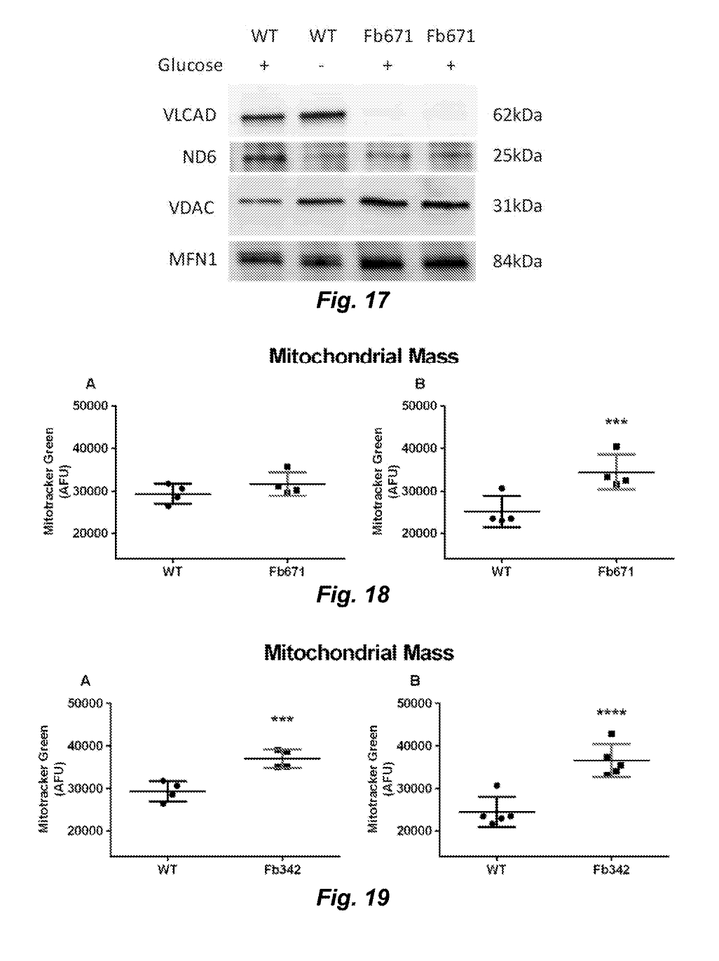

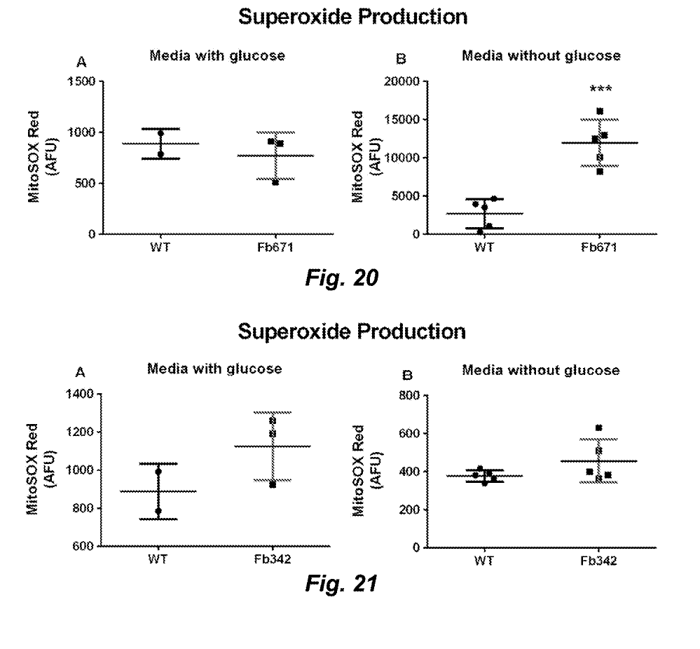

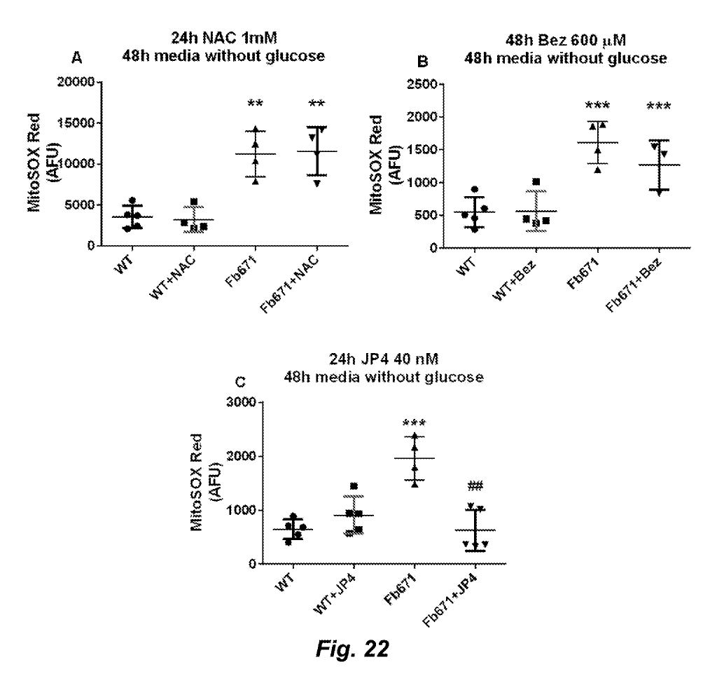

VLCAD- and MCAD-deficient fibroblasts showed a decrease of basal respiration and reserve capacity as measured by oxygen consumption (FIGS. 11-14), compared to control (WT). ATP production was decreased 48 h after treating the cells with medium without glucose (FIGS. 15 and 16). We also observed absence of VLCAD protein, increased VDAC₁ protein and a decrease of complex I subunit ND6 in VLCAD deficient fibroblasts (FIG. 17). Mitochondrial mass was increased in both cell lines (FIGS. 18 and 19), and superoxide production was increased in VLCAD deficient fibroblasts (FIGS. 20 and 21), which was prevented by the mitochondrially targeted ROS scavenger JP4-039, but not by bezafibrate and N-acetylcysteine (FIG. 22).

These findings identify increased ROS production with disrupted long chain FAO in VLCAD deficient fibroblasts cultivated in the absence of glucose. In addition, respiratory chain function is decreased in VLCAD and MCAD deficient fibroblasts, indicating that the energy metabolism dysfunction in VLCAD deficiency exceeds that of FAO alone. We also verified increased mitochondrial mass in the VLCAD fibroblasts cultivated in the absence of glucose, as well as in LCAD fibroblasts. These novel pathophysiological mechanisms in FAO disorders suggest new possibilities for therapeutic strategies.

These findings identify decreased respiratory chain function in MCAD and VLCAD deficient fibroblasts, indicating that the energy metabolism dysfunction exceeds that of FAO alone. In addition, mitochondrial mass is increased in both, while VLCAD deficient fibroblasts also present increased ROS production. These pathophysiological manifestations in these disorders suggest new possibilities for therapeutic strategies.

### Example 3

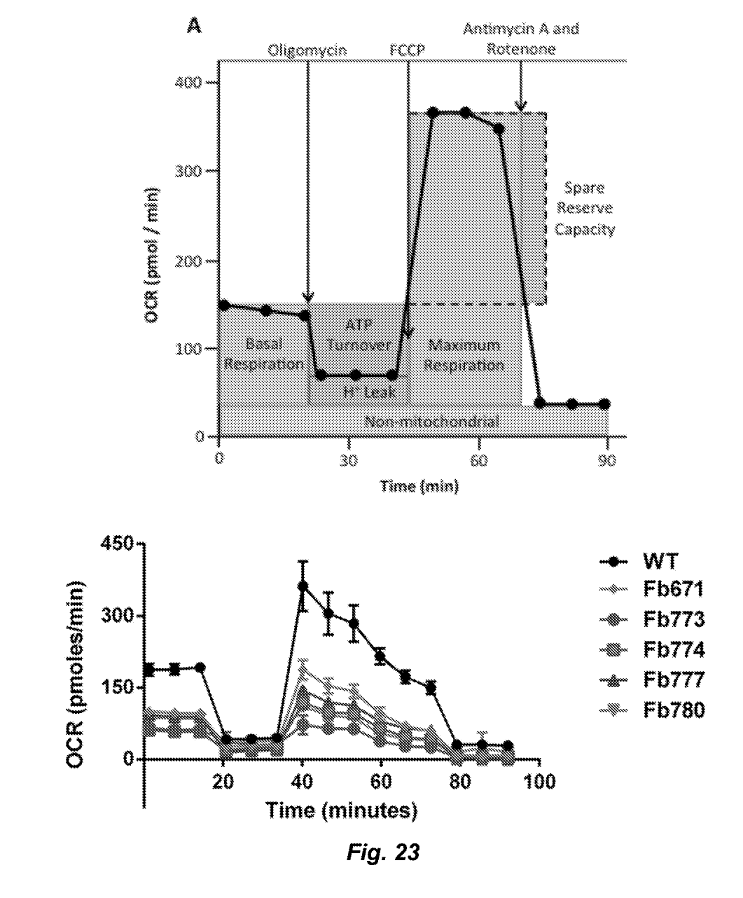

FAO deficient fibroblasts were cultured in the absence of glucose for 48-72 h to shift to fatty acids as the energy source. Oxygen consumption rate (OCR) was monitored by a Seahorse XF analyzer (See FIG. 23). Mitochondrial mass was examined by MitoTracker Green and ROS generation by MitoSOX dye. Protein immunocontent was evaluated by western blotting. FAO deficient cells showed decreased basal respiration and reserve capacity and increased superoxide production, which was prevented by the mitochondrial targeted electron, radical, or ROS-scavenging agents JP4-039 and XJB-5-131, but not by a standard antioxidant N-acetylcysteine. Testing of XJB-5-131 was conducted essentially as described for JP4-039.

### Materials and Methods

Subjects.

Fibroblast cells with mutation in FAO gene were obtained from patients' skin biopsies, while control fibroblast cells (wild type; WT) were obtained from three healthy individuals. Biopsies from patients were performed on a clinical basis with written informed consent from patients and/or parents, or from anonymous donors.

Cell Culture and Treatments.

Cells were grown in Dulbecco's Modified Eagle Medium (DMEM), Corning Life Sciences, Manassas, Va., containing high glucose levels or in DMEM devoid of glucose for 48-72 h. Both media were supplemented with glutamine, 10% fetal bovine serum, 4 mM glutamine, 100 IU penicillin and 100 μg/mL streptomycin, Corning Life Sciences, Manassas, Va.

JP4-039 Electron, Radical, or ROS-Scavenging Agent Treatment.

Cells were treated with different compounds in different concentrations 24 or 48 h before the assays. The compounds used were N-acetylcysteine (1 mM), bezafibrate (600 μM), resveratrol (75 μM), MiloQ (200 nM), trolox (1 mM) and JP4-039 (40 and 200 nM).

Measurement of Mitochondrial Respiration.

Oxygen consumption rate (OCR) was measured with a Seahorse XF^{c}96 Extracellular Flux Analyzer (Seahorse Bioscience, Billerica, Mass.). The apparatus contains a fluorophore that is sensitive to changes in oxygen concentration, which enables it to accurately measure the rate at which cytochrome c oxidase (complex IV) reduces one O₂ molecule to two H₂O molecules during OXPHOS. Cells were seeded in 96-well Seahorse tissue culture microplates in growth media at a density of 80,000 cells per well. To ensure equal cell numbers, cells were seeded in cell culture plates pre-coated with Cell-Tak (BD Biosciences, San Jose, Calif.). All cell lines were measured with four to six wells per cell line. Then, the entire experiments were repeated. Before running the Seahorse assay, cells were incubated for 1 h without CO₂ in unbuffered DMEM. Initial OCR was measured to establish a baseline (basal respiration). Reserve capacity was also determined after the injection of 300 nM carbonyl cyanide 4-(trifluoromethoxy) phenylhydrazone (FCCP), Seahorse XF Cell Mito Stress Test Kit, Santa Clara, Calif. Data were reported in pmol/min for OCR.

ATP Production Assay.

ATP production was determined by a bioluminescence assay using an ATP determination kit (ATPlite kit) from PerkinElmer Inc, Waltham, Mass., according to the manufacturer's instructions. The luminescence was measured in a FLUOstar Omega plate reader (BMG Labtech), Ortenberg, Germany.

Mitochondrial Membrane Mass and Superoxide Production.

Cell suspension containing 1×10⁵ cells per mL were incubated for 25 min at 37° C. with 150 nM Mitotracker Green, Invitrogen, Grand Island, N.Y., for mitochondrial mass evaluation or for 15 min at 37° C. with 5 μM MitoSOX Red, Invitrogen, Grand Island, N.Y., for superoxide production measurement. After incubation, 10,000 cells samples were analyzed in a Becton Dickinson FACSAria II flow cytometer, BD Biosciences, San Jose, Calif.

Western Blot.

Cells were grown in T175 flasks and, at 90-95% confluence, were harvested by trypsinization, pelleted and stored at −80° C. for western blot. Protein content in samples was quantified for data normalization using DC™ Protein Assay kit (Bio-Rad Laboratories).

Mitochondria Preparation.

Cell pellets were re-suspended in 150-250 μL of 5 mM Tris buffer, pH 7.4, containing 250 mM sucrose, 2 mM EDTA, protease inhibitor cocktail, Roche Diagnostics, Mannheim, Germany, and 0.5 μM trichostatin A, Sigma-Aldrich Co., St. Louis, Mo., homogenized and centrifuged at 1,000 g for 5 min at 4° C. The pellet was discarded and the supernatant centrifuged at 12,000 g for 15 min at 4° C. The pellet containing mitochondria was re-suspended in 50 mM Tris buffer, pH 7.4, sonicated and centrifuged again at 14,000 g for 15 min at 4° C. The supernatant was then used for western blotting. Briefly, 10-20 μg of protein was loaded onto the gel. Following electrophoresis, the gel was blotted onto a nitrocellulose membrane, which was incubated with rabbit mouse anti-mitofusin 1 monoclonal antibody (MFN1; 1:100), Abcam, Cambridge, Mass., rabbit anti-VLCAD (very long-chain acyl-CoA dehydrogenase) antiserum (1:1,000), Cocalico Biologicals Inc., PA, Cocalico Biologicals Inc., rabbit anti-VDAC/Porin monoclonal antibody (1:1,000), Abcam, Cambridge, Mass., or IgG-HRP conjugated antibody, Bio-Rad, Hercules, Calif. Staining of the membranes with Ponceau S, Sigma-Aldrich Co., St. Louis, Mo., was used to verify equal loading.

Nuclear and Cytosolic Fraction Preparation.

Cells pellets were washed with cold phosphate-buffered saline (PBS) and lysed with a pre-cooled homogenizer in 300 μL cold buffer (10 mM HEPES, 1.5 mM MgCl₂, 1 mM KCl and 1 mM DTT) plus 1 μg/μL protease, phosphatase inhibitor cocktail, 1 mM PMSF and 0.5% Nonidet P-40, and incubated on ice for 15 min. The homogenates were centrifuged at 850×g for 10 min at 4° C., and the supernatants (cytoplasmic extracts, SN1) were collected and stored at −80° C. The pellets were resuspended in 200 μL of cold buffer, transferred to pre-cooled microcentrifuge tubes and incubated on ice for 15 min. Then, 0.5% Nonidet P-40 was added and the samples were incubated on ice for 5 min and mixed for 10 s. The suspensions were centrifuged at 14,000×g for 30 s at 4° C., and the supernatants were collected in SN1. Then, the pellets were resuspended in 50 μL of complete lysis buffer (20 mM HEPES, 1.5 mM MgCl₂, 0.2 mM EDTA, 20% glycerol, 420 mM NaCl and 1 mM DTT), plus 1 μg/mL protease, phosphatase inhibitor cocktail, and 1 mM PMSF, mixed for 10 s, and incubated on ice for 40 min (mixed for 10 s each 5 min). Finally, the suspensions were mixed for 30 s and centrifuged at 14,000×g for 10 min at 4° C. The supernatants (nuclear extracts, SN2) were collected and stored at −80° C. Inmunodetection was performed using the following primary antibodies, according to datasheet specifications: anti-Nrf2 (1:500), Abcam®, Cambridge, Mass., ab31163, anti-NF-κB p-65 (1:500), Abcam®, Cambridge, Mass., ab32536, anti-Lamin B1 (1:1000) Abcam®, Cambridge, Mass., ab133741, and anti-β-actin (1:1000) Santa Cruz Biotechnology®, Dallas, Tex.,

Immunofluorescence Microscopy.

Fibroblasts were seeded at a concentration of 1×10⁵ cells/mL on glass cover slips pre-treated with poly-D-lysine in a 12-well plate and allowed to grow overnight at 37° C. in a 5% CO₂/95% humidity incubator. After 80-90% confluence, cells were incubated with the antibodies anti-VLCAD (1:1,000), anti-Nrf2 (1:100) or anti-NF-κB (1:1,000) at 4° C. overnight. After brief washing with TBST, cells were incubated with donkey anti-rabbit secondary antibody Alexa Fluor 488, from Invitrogen. Nuclei were immunostained with DAPI. The coverslips were then mounted using mounting media before taking images with an Olympus Confocal FluoroView1000 microscope at a magnification of 60×. Mitochondrial membrane potential was determined by quantification of MitoTracker Red fluorescence, from Invitrogen, using the software ImageJ, Bethesda, Md., and the data were normalized by number of cells.

Fatty Acid Oxidation (FAO) Flux Analysis.

FAO analysis flux was performed by quantifying the production of ³H₂O from 9,10-^{[3H]}palmitate, PerkinElmer, Waltham, Mass., conjugated to fatty acid-free albumin in fibroblasts cultured in a 24-well plate. Palmitate bound to albumin was used at the final concentration of 12.4 μM (0.06 Ci/mmol). For each cell line, FAO measurement was performed in triplicate. The oxidation rates were expressed as pmol ³H-fatty acid oxidized/h/mg protein).

Cell Viability Assay.

Cell viability was evaluated according to the instructions described in MTS assay kit from Abcam, Cambridge, UK, according to the manufacturer's instructions. The absorbance was read in the FLUOstar Omega plate reader at 490 nm.

Spectrophotometric Analysis of Citrate Synthase Activity.

Citrate synthase (CS) activity was measured in mitochondria obtained from fibroblast by measuring DTNB reduction at k=412 nm, and calculated as nmol TNB min⁻¹ mg protein⁻¹.

Statistical Analysis.

Assays were performed in triplicate and the mean was used for statistical calculations. Duplicate or triplicate experiments were always carried out and the mean used for the calculations. Statistical analysis was performed with GraphPad 5.0 software. Student's 1 test (independent) was applied for simple comparisons between groups. Differences were considered significant when P<0.05. Only significant results are presented.

### Results

Oxygen Consumption and ATP Production.

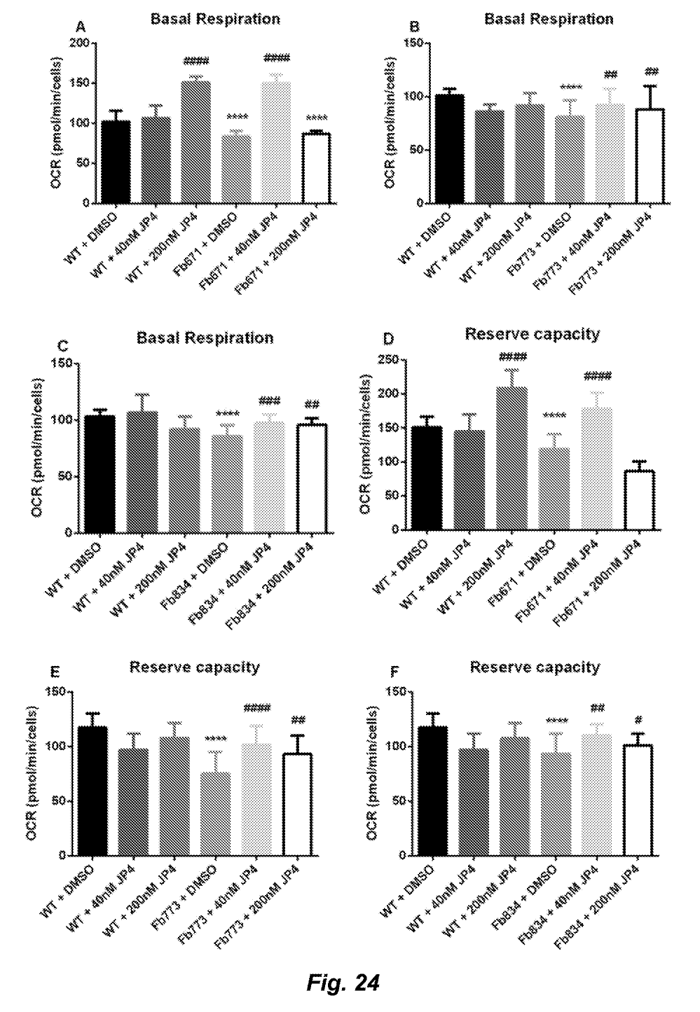

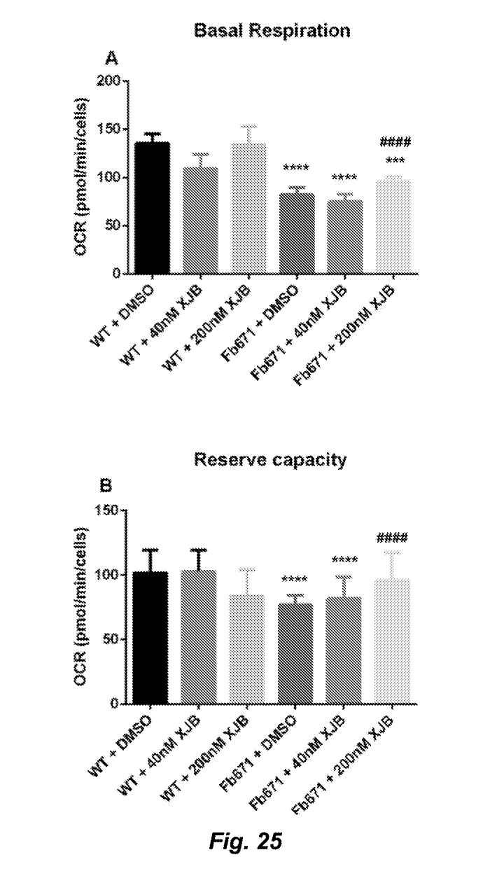

The bioenergetics state of the patients' fibroblasts was measured by monitoring oxygen consumption in Seahorse analyzer. It was observed that basal respiration and reserve capacity (FIG. 24) were decreased in all VLCAD deficient fibroblasts in media without glucose. FIG. 25 provides results for XJB-5-131 in media without glucose. Patient Fb671 was also tested using normal media, and the same decrease was detected (FIGS. 11 and 12 (Example 2)). Some of the patient's fibroblasts were also treated with JP4-039 for 24 hours and basal respiration and reserve capacity were measured. We verified that this electron, radical, or ROS-scavenging agent significantly increased both parameters (FIG. 24).

Next we determined ATP production to assess the consequences of reduced oxygen consumption. As shown in FIG. 15 (Example 2), a marked reduction of ATP levels in VLCAD cells. Taken together, this data show energy homeostasis impairment in VLCAD deficient fibroblasts.

Mitochondrial Mass and ΔΨ.

Mitochondrial mass using MitoTracker green was evaluated in VLCAD-deficient fibroblasts. FIG. 18 (Example 2) shows an increase in mitochondrial mass in Fb671 fibroblasts, whereas no alterations were verified when cell lines were maintained in media with glucose.

Mitochondrial Dynamics and Interaction with ETC.

As the VLCAD-deficient cells had mitochondrial mass alterations, we measured the protein levels of MFN1 and ND6 subunit, the main molecule implicated in fusion and a complex I subunit, respectively. MFN-1 content was increased in Fb671 fibroblasts cultured media with glucose and without glucose as compared to control cells. Regarding ND6 subunit, we found an increase of this protein levels in both conditions (FIG. 17 (Example 2)).

Superoxide Production.

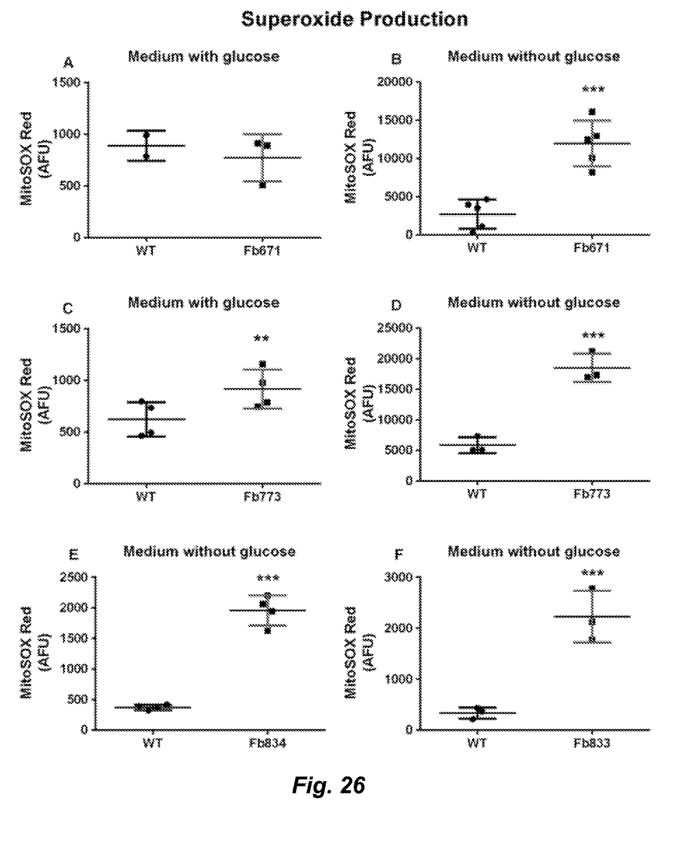

Since mitochondrial dysfunction may have the involvement of reactive species generation, we determined superoxide levels with the probe MitoSOX Red in VLCAD deficient fibroblasts (Fb671, Fb773, Fb834 and Fb833). Significant alterations were observed in all VLCAD cell lines tested, in media with or without glucose (FIG. 26).

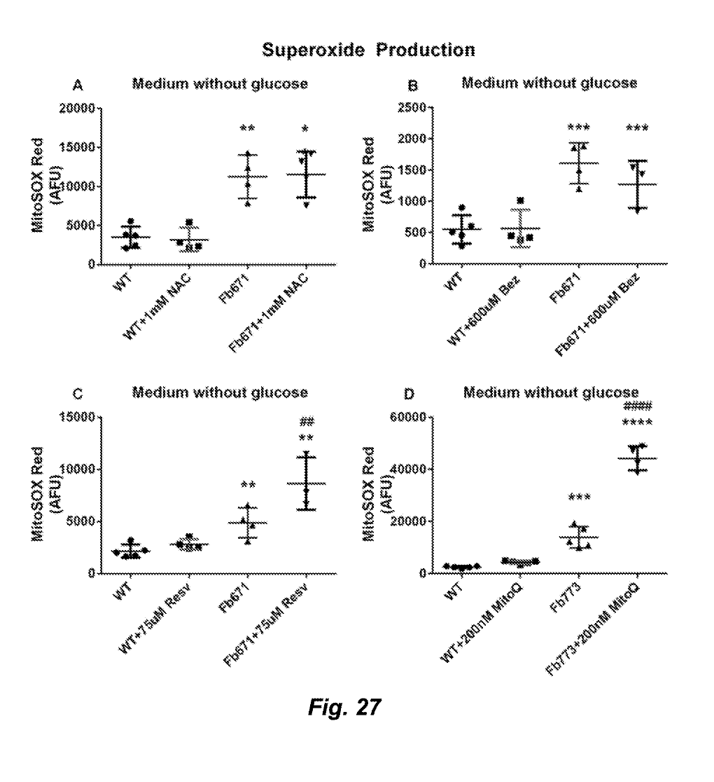

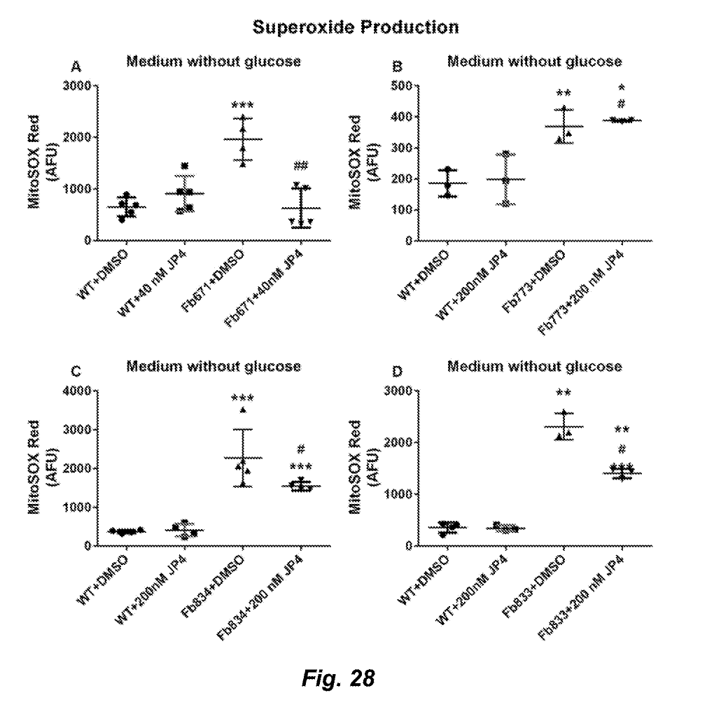

The effect of the presence of bezafibrate, N-acetylcysteine, resveratrol, MitoQ, and JP4-039 in the media as antioxidants on the increased levels of superoxide detected in VLCAD deficient cells was also tested. JP4-039 was the only antioxidant able to decrease superoxide generation in the patient cells after 24 h of treatment (FIGS. 27 and 28).

Protein Expression of the Transcription Factors Nrf2 and NF-κB.

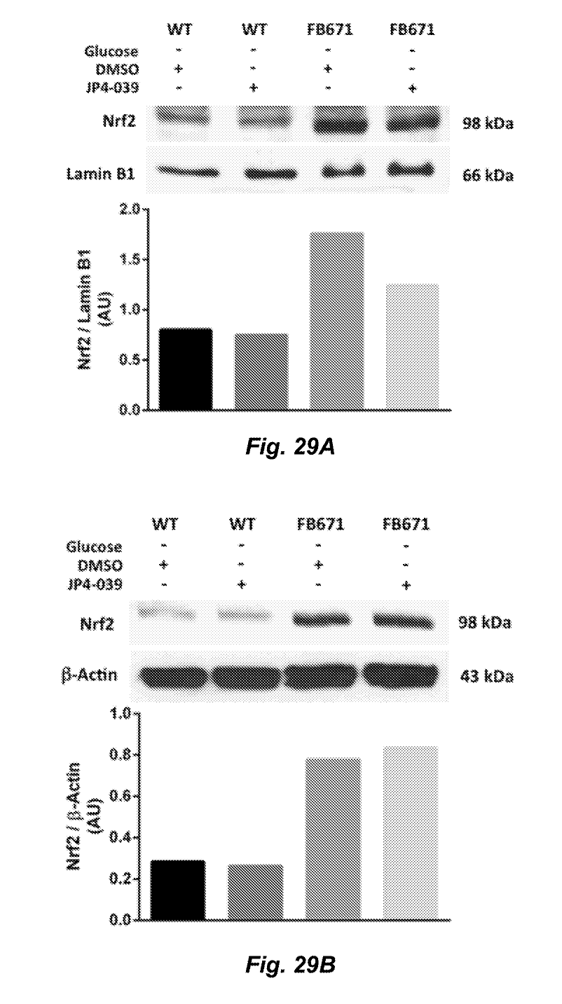

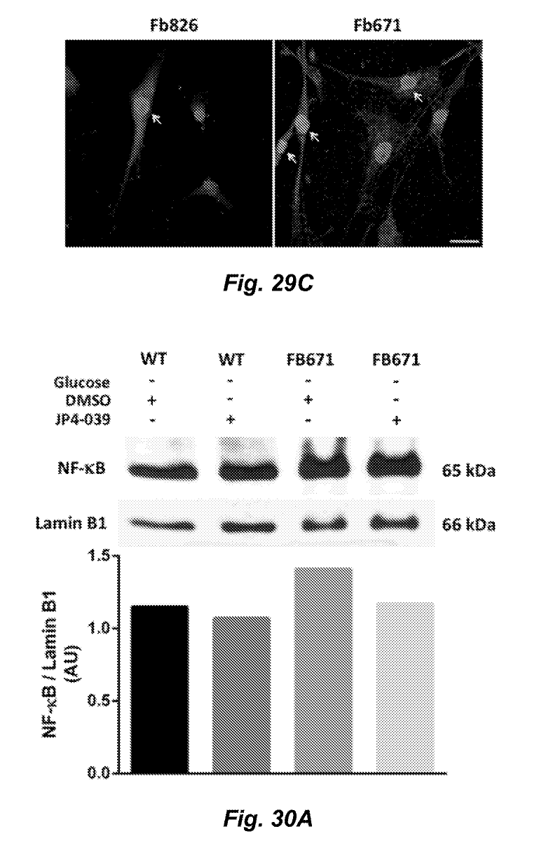

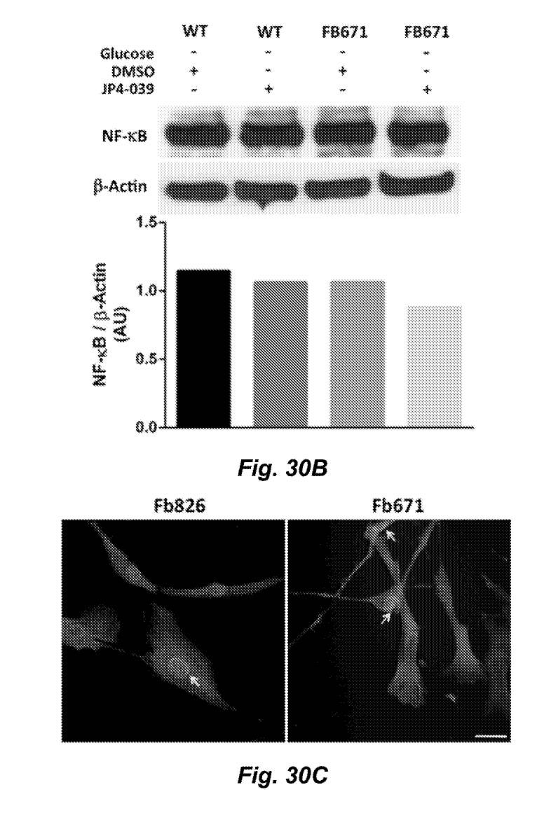

After observing evidence for oxidative stress in the VLCAD patients' fibroblasts, we evaluated the protein content of two important transcription factors involved in redox homeostasis, after treating the cells with JP4-039. Increased nuclear Nrf2 and NF-κB protein levels were observed in the Fb671 fibroblasts, when compared to the WT fibroblasts (FIGS. 29A, 29B, 29C, 30A, 30B, and 30C). The cytosolic content of Nrf2 was also increased in VLCAD deficient fibroblasts comparing to the control, while the NF-κB cytosolic levels were not altered. The treatment with JP4-039 slightly decreased the elevated protein expression of these transcription factors, in the nucleus, as well as in the cytosol for the NF-κB (FIG. 30B).

FAO Flux and VLCAD Content.

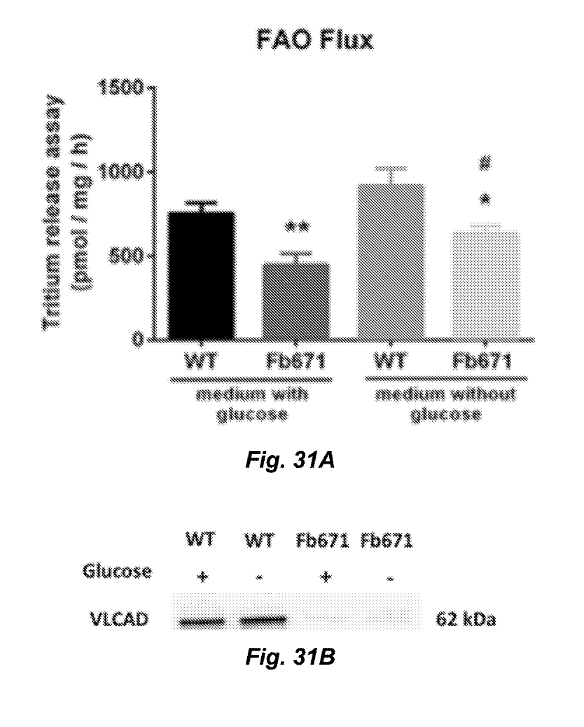

The flux through the FAO pathway in Fb671 fibroblasts was further investigated. Decreased FAO flux was verified in cells in the presence of glucose comparing to control cells, while a slight but significant increase was observed in in media without glucose (FIG. 31A). Then, VLCAD protein content was examined to correlate with the FAO flux alterations observed. The protein expression of VLCAD was initially evaluated in fibroblasts cultured in media with or without glucose for 48 h. FIG. 31B shows that the absence of glucose caused an increase of VLCAD in WT cells. Decreased VLCAD presence was also observed in VLCAD deficient cells in media with glucose as compared to WT cells. And finally, the absence of glucose increased the VLCAD content, correlating with the FAO flux study.

Cell Viability and VDAC1/Porin Protein Content.

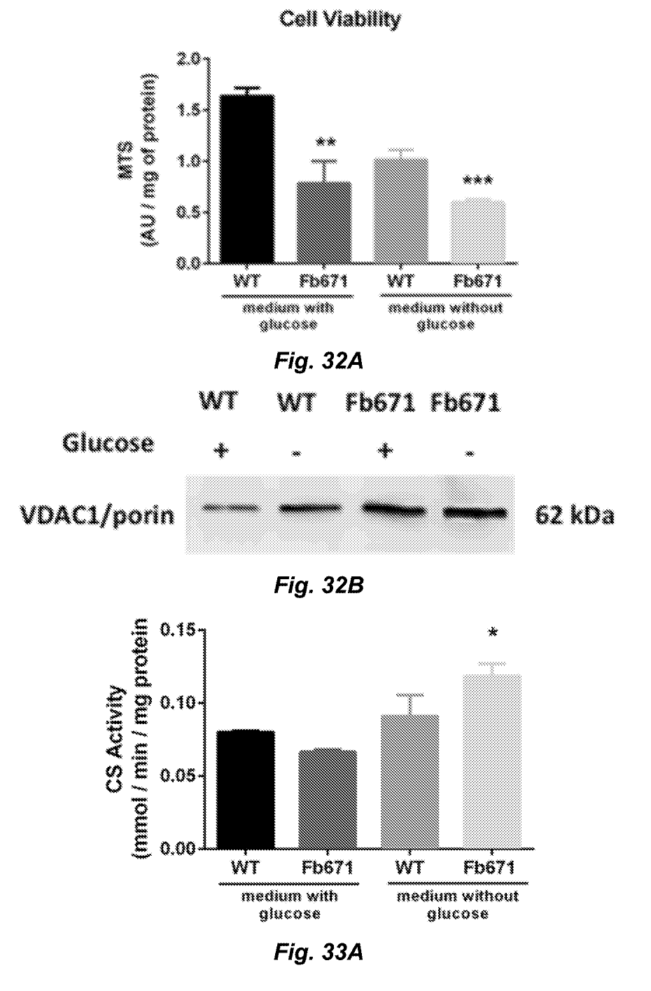

We evaluated cell viability by using MTS reduction assay in the VLCAD-deficient cells. Decreased cell viability was verified in fibroblasts in media with or without glucose (FIG. 32A). We further determined the content of VDAC1/porin (FIG. 32B), a multifunctional mitochondrial channel that mediates the mitochondrial transfer of metabolites, and is involved in transition pore formation and apoptosis. Increased VDAC1/porin content was observed in Fb671 fibroblasts, in media with or without glucose, comparing to the WT cells (FIG. 32C).

Citrate Synthase Activity.

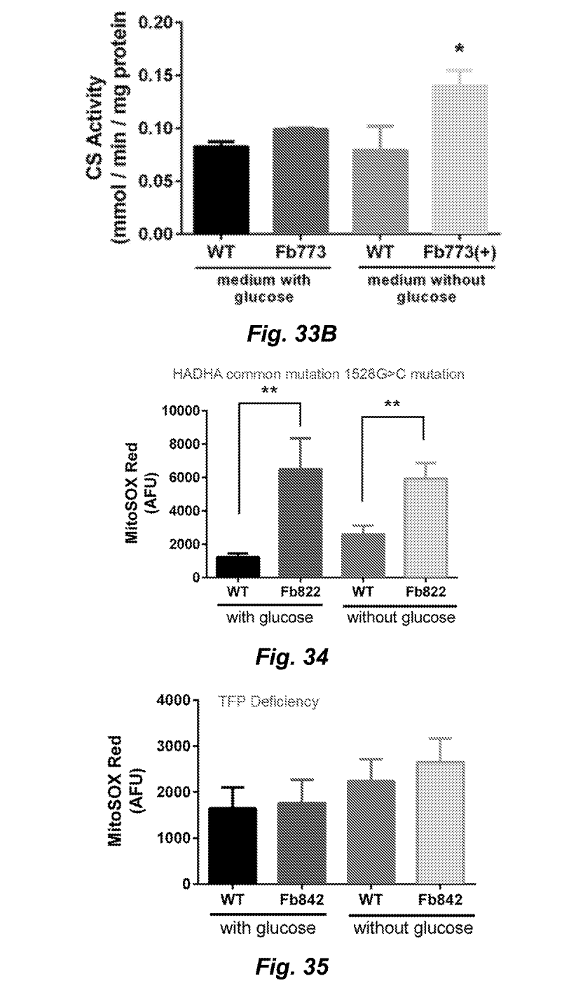

Citrate synthase activity was measured in Fb671 and Fb773 fibroblasts. Comparing to the control cell line, both patients' cells presented an increased of this enzyme activity after being cultured in media avoided from glucose for 48 h (FIGS. 33A and 33B).

Additional Cell Measurements.

LCHAD deficient, MTP (TFP) deficient (with a mutation in HADHB), and additional VLCAD deficient cell lines were tested as above and all showed defects in oxygen consumption and increased accumulation of reactive oxygen species. Treatment with JP4 improved both parameters (FIGS. 34 and 35).

In sum, mitochondrial targeted electron, radical, or ROS-scavenging agents were effective in reducing accumulation of superoxide species in cells from patients with FAO defects and improving functional measurements of bioenergetics of these cells.

1. A method of treating a fatty acid oxidation or respiratory chain metabolic condition, and/or hypoglycemia, rhabdomyolysis, lactic acidosis, hyperammonemia, skeletal myopathy, hypotonia, fatiguabilty, or cardiomyopathy in a patient, comprising administering to the patient an amount of a mitochondria-targeting electron, radical, or ROS-scavenging agent effective to treat the fatty acid oxidase metabolic condition, and/or hypoglycemia, rhabdomyolysis, or cardiomyopathy in a patient.

2. The method of claim 1, wherein the mitochondria-targeting electron, radical, or ROS-scavenging agent is a compound comprising an electron, radical, or ROS-scavenging moiety and a mitochondria-targeting moiety.

3. The method of claim 2, wherein the compound comprises an electron, radical, or ROS-scavenging moiety and a mitochondria-targeting moiety has a structure chosen from:
a. 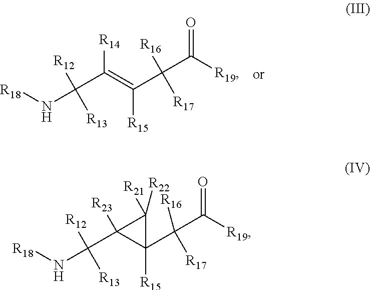
wherein R₁₂, R₁₃, R₁₆, and R₁₇ are independently hydrogen, hydroxyl, halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted, for example, and without limitation, R₁₂, R₁₃, R₁₆, and R₁₇ are independently methyl-, hydroxyl- or fluoro-substituted, including: methyl, ethyl, propyl, 2-propyl, butyl, t-butyl, pentyl, hexyl, benzyl, hydroxybenzyl (e.g., 4-hydroxybenzyl), phenyl, or hydroxyphenyl;
R₁₅ is hydrogen, a halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted;
R₁₈ is —C(O)—R₂₄, —C(O)O—R₂₄, or —P(O)—(R₂₄)₂, wherein R₂₄ is C₁-C₆ straight or branched-chain alkyl or a C₁-C₆ straight or branched-chain alkyl optionally comprising one or more (C₆H₅) groups that are independently unsubstituted, or methyl-, ethyl-, hydroxyl-, halo-substituted or fluoro-substituted, for example and without limitation, R₁₈ is Ac (Acetyl, R=—C(O)—CH₃), Boc (R=—C(O)O-tert-butyl), Cbz (R=—C(O)O-benzyl (Bn)), or a diphenylphosphate group;
R₁₉ is —NH—R₂₀, —O—R₂₀ or —CH₂—R₂₀, where R₂₀ is an electron, radical, or ROS-scavenging moiety, such as an N—O., N—OH, or N═O-containing moiety, e.g., a nitroxide-containing moiety, that can be covalently attached by any useful chemistry to a mitochondria-targeting moiety, such as shown for XJB-5-131;
R₁₄ is a halo, a C₁-C₆ straight or branched-chain alkyl or a C₁-C₆ straight or branched-chain alkyl further comprising one or more (C₆H₅) groups that are independently unsubstituted, or methyl-, ethyl-, hydroxyl- or halo-substituted; and
R₂₁, R₂₂, and R₂₃ are independently H or a halo;
b. 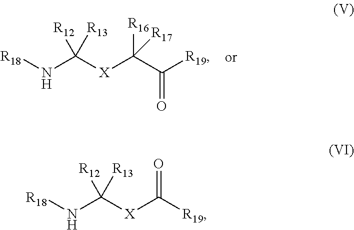
wherein X is 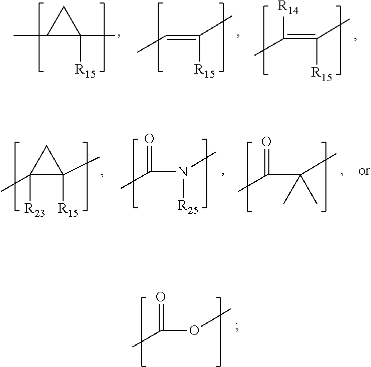
R₁₂, R₁₃, R₁₆, R₁₇, and R₂₅ are each independently hydrogen, halo, a C₁-C₆ straight or branched-chain alkyl, or a C₁-C₆ straight or branched-chain alkyl further comprising a phenyl (C₆H₅) group, wherein the C₁-C₆ straight or branched-chain alkyl group or the C₁-C₆ straight or branched-chain alkyl group comprising a phenyl group is unsubstituted or is methyl-, hydroxyl- or halo-substituted;
R₁₉ is —NH—R₂₀, —O—R₂₀ or —CH₂—R₂₀, where R₂₀ is an electron, radical, or ROS-scavenging moiety, such as an N—O., N—OH, or N═O-containing moiety, e.g., a nitroxide-containing moiety, that can be covalently attached by any useful chemistry to a mitochondria-targeting moiety, such as shown for XJB-5-131; and
R₁₈ is —C(O)—R₂₄, —C(O)O—R₂₄, or —P(O)—(R₂₄)₂, wherein R₂₄ is C₁-C₆ straight or branched-chain alkyl or a C₁-C₆ straight or branched-chain alkyl optionally comprising one or more (C₆H₅) groups that are independently unsubstituted, or methyl-, ethyl-, hydroxyl-, halo-substituted or fluoro-substituted, for example and without limitation, R₁₈ is Ac (Acetyl, R=—C(O)—CH₃), Boc (R=—C(O)O-tert-butyl), Cbz (R=—C(O)O-benzyl (Bn)), or a diphenylphosphate group; or
c. 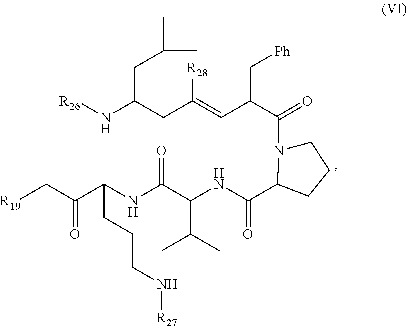
wherein R₁₉ is —NH—R₂₀, —O—R₂₀ or —CH₂—R₂₀, where R₂₀ is an electron, radical, or ROS-scavenging moiety, such as an N—O., N—OH, or N═O-containing moiety, e.g., a nitroxide-containing moiety, that can be covalently attached by any useful chemistry to a mitochondria-targeting moiety, such as shown for XJB-5-131; R₂₆ and R₂₇, independently are an amine protecting group or acylated. In one aspect, R₂₆ and R₂₇ are protecting groups independently selected from the group consisting of: 9-fluorenylmethyloxy carbonyl (Fmoc), t-butyloxycarbonyl (Boc), benzhydryloxycarbonyl (Bhoc), benzyloxycarbonyl (Cbz), O-nitroveratryloxycarbonyl (Nvoc), benzyl (Bn), allyloxycarbonyl (alloc), trityl (Trt), 1-(4,4-dimethyl-2,6-dioxacyclohexylidene)ethyl (Dde), diathiasuccinoyl (Dts), benzothiazole-2-sulfonyl (Bts), dimethoxytrityl (DMT) and monomethoxytrityl (MMT), and R₂₈ is H or methyl. In one aspect, R₂₆ is Boc and R₂₇ is Cbz. Ph is phenyl,
or a pharmaceutically-acceptable salt or ester thereof.

4. The method of claim 3, having the structure of (III) or (IV), or a pharmaceutically-acceptable salt or ester thereof.

5. The method of claim 3, having the structure of (V) or (VI), or a pharmaceutically-acceptable salt or ester thereof.

6. The method of claim 3, having the structure of (VII), or a pharmaceutically-acceptable salt or ester thereof.

7. The method of claim 3, having the structure: 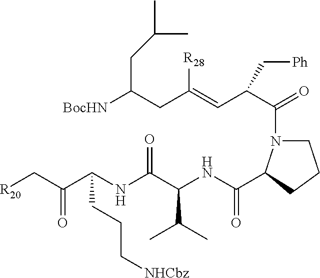
wherein R₁₉ is —NH—R₂₀, —O—R₂₀ or —CH₂—R₂₀, where R₂₀ is an electron, radical, or ROS-scavenging moiety, such as an N—O., N—OH, or N═O-containing moiety, e.g., a nitroxide-containing moiety, that can be covalently attached by any useful chemistry to a mitochondria-targeting moiety, such as shown for XJB-5-131, or a pharmaceutically-acceptable salt or ester thereof.

8. The method of claim 1, wherein the mitochondria-targeting electron, radical, or ROS-scavenging agent is chosen from: XJB-5-125, XJB-5-131, XJB-5-197, XJB-7-53, XJB-7-55, XJB-7-75, JP4-039 and JP4-049.

9. The method of claim 1, wherein the mitochondria-targeting electron, radical, or ROS-scavenging agent is JP4-039.

10. The method of claim 1, wherein the mitochondria-targeting electron, radical, or ROS-scavenging agent is XJB-5-131.

11. The method of claim 1, wherein the fatty acid oxidase metabolic condition, and/or hypoglycemia, rhabdomyolysis, or cardiomyopathy is an inborn error of fatty acid oxidation or oxidative phosphorylation.

12. The method of claim 11, wherein the inborn error of fatty acid oxidation or oxidative phosphorylation is chosen from defects in the following enzymes: carnitine palmitoyltransferase (CPT) I; CPT II; carnitine-acylcarnitine translocase (CACT); very long-chain acyl-CoA dehydrogenase (VLCAD); medium-chain acyl-CoA dehydrogenase (MCAD); and long-chain hydroxyacyl-CoA dehydrogenase (LCHAD).

13. (canceled)

14. (canceled)
